(19) United States
(12) Patent Application Publication
(10) Pub. No.: US 2025/0082453 A1
(43) Pub. Date: Mar. 13, 2025

(54) METHODS, SYSTEMS AND COMPUTER PROGRAM PRODUCTS FOR DELIVERING A SUBSTANCE TO A SUBJECT

(71) Applicant: TARGAN Inc., Raleigh, NC (US)

(72) Inventors: Joshua Grenon, Chapel Hill, NC (US); Jonathan Adams, Pittsboro, NC (US)

(73) Assignee: TARGAN Inc., Raleigh, NC (US)

(21) Appl. No.: 18/291,632

(22) PCT Filed: Aug. 16, 2022
(86) PCT No.: PCT/US2022/075004

§ 371 Date: Jan. 24, 2024

Related U.S. Application Data
(60) Provisional application No. 63/234,034, filed on Aug. 17, 2021.

(51) Int. Cl. A61D 7/00; G06T 7/20; G06T 7/70

(52) U.S. Cl. CPC A61D 7/00; G06T 7/20; G06T 7/70; G06T 2207/30004

(57) ABSTRACT

A method for accurately administering a substance to a subject in motion is provided including obtaining one or more scans of the subject. The subject has at least one defined target region thereon for delivery of the substance. A three dimensional position of the subject in motion is calculated based on the obtained one or more scans of the subject. The three dimensional position includes X, Y, and Z coordinates defining the three dimensional position. A timing adjustment is calculated based on the calculated three dimensional position of the subject in motion. A timing of the delivery of the substance to the at least one defined target region is adjusted based on the subject using the calculated timing adjustment. The obtaining, calculating the three dimensional position, calculating the timing adjustment and the adjusting are performed by at least one processor.

### CLAIM OF PRIORITY

The present application claims the benefit of and priority to U.S. Provisional Patent Application No. 63/234,034 filed on Aug. 17, 2021, entitled Methods, Systems and Computer Program Products for Delivering a Substance to a Subject, the entire content of which is incorporated herein by reference.

### FIELD

The present inventive concept relates generally to delivery of a substance to a subject and, more particularly, accommodating for movement of the subject in three dimensions while delivering a substance.

### BACKGROUND

Bacterial, viral and fungal infections and other diseases are often treated through vaccination, or delivery of a drug to a subject. In all animals, and in particular, vertebrates or fish, and invertebrates, such as crustaceans, the delivery of vaccines, biologics and other medicine is often delivered to reduce the likelihood of disease or death or to maintain overall good health. In many livestock and fish operations, it is a challenge to ensure that all animals have been effectively treated. The number and variation in the size of the subject makes vaccination and delivery of other medicine to each subject a challenge.

For example, vaccination of poultry can be particularly difficult due to the size of the poultry at the time of vaccination as well as the number of animals being vaccinated during a single time period. Currently, poultry may be vaccinated while still inside the egg or the chicks may be treated after hatching. Specifically, these methods may include automated vaccination in the hatchery performed “in ovo” (within the egg) on day 18 or 19; automated mass vaccination in the hatchery performed “post-hatch”; manual vaccination in the hatchery performed “post-hatch”; vaccination/medication added to the feed or water in the “Growth Farm”; and vaccination/medication sprayed on the chicks either manually or by mass sprayers.

While the poultry industry spends over $3 billion on vaccines and other pharmaceuticals on an annual basis, the return on their investment is not guaranteed due to the challenges with the manner in which the vaccines or other substances are delivered. Each aforementioned method has shown noticeable and significant inadequacies. Thus, an automatic system and method for delivering vaccination to animals has been developed as discussed in, for example, PCT publication No. WO 2017/083663, the disclosure of which is hereby incorporated herein by reference. However, even the automatic system did not ensure that each animal has received an effective dose of the vaccine.

### SUMMARY

Some embodiments of the present inventive concept provide a method for accurately administering a substance to a subject in motion, the method including obtaining one or more scans of the subject. The subject has at least one defined target region thereon for delivery of the substance. A three dimensional position of the subject in motion is calculated based on the obtained one or more scans of the subject. The three dimensional position includes X, Y, and Z coordinates defining the three dimensional position. A timing adjustment is calculated based on the calculated three dimensional position of the subject in motion. A timing of the delivery of the substance to the at least one defined target region is adjusted based on the subject using the calculated timing adjustment. The obtaining, calculating the three dimensional position, calculating the timing adjustment and the adjusting the delivery timing are performed by at least one processor.

In further embodiments only a single scan of a whole subject in motion may be obtained.

In still further embodiments, a first slice scan of the subject in motion may be obtained. The first slice scan is a scan of less than a whole subject. It is determined that the first slice scan exceeds a threshold indicating that an entire defined target area is visible in the first slice scan. If it is determined that the entire defined target area is visible in the first slice scan, the three dimensional position of the subject in motion is calculated based on the first slice scan. If it is determined that the first slice scan does not exceed the threshold, an additional slice scan may be obtained. The first slice scan and the additional slice scan may be combined to provide a combined scan. It is determined if the combined scan exceeds the threshold. The obtaining and combining steps are repeated until it is determined that the threshold has been exceeded and then the three dimensional position of the subject in motion is calculated based on the combined scan when it is determined that the threshold indicating that the entire defined target area is visible is exceeded.

In some embodiments, the method may further include calculating a nozzle adjustment factor based on the calculated three dimensional position of the subject in motion. A position of at least one nozzle used to administer the substance may be adjusted based on the calculated nozzle adjustment factor.

In further embodiments, calculating the timing adjustment and the nozzle adjustment factor may include calculating the timing adjustment and the nozzle adjustment factor based on one or more of the following: a velocity of a conveyor belt on which the subject is traveling (v_{b}); a time of flight (TofF) before the substance is delivered to the subject; a speed at which the substance is delivered (vₛ); a distance the at least one defined target region is from a nozzle delivering the substance (dₜₙ); and a width of the conveyor belt (w_{c}).

In still further embodiments, the substance may be administered to the at least one defined target region of the subject at a time and position altered by the nozzle adjustment factor and/or the timing adjustment.

In some embodiments, the at least one nozzle may be one or more nozzle banks.

In further embodiments, the subject may be a bird and the at least one defined target region may be mucosa in one or more eyes of the bird, an area around one or more eyes of the bird, nostrils of the bird, mouth of the bird, and/or any orifice on a head of the bird that leads to the gut and/or the respiratory tract.

In some embodiments, the subject maybe a swine. In these embodiments, the method may further include delivering the substance to the swine using at least one needle or needle free injector.

In further embodiments, the substance may be delivered in a volume no greater than 120 ul/subject.

In still further embodiments, the method may further include delivering the substance to the subject from a day of hatch to chicks having an age of five days.

In some further embodiments, the subject may be any human or animal that receives the substance.

In further embodiments, at least 85% of the subjects receive delivery of the substance in the at least one defined target region.

In still further embodiments, greater than 92% of the subjects receive delivery of the substance in the at least one defined target region.

### BRIEF DESCRIPTION OF THE DRAWINGS

FIG. 1A is a basic block diagram illustrating a system including a location module in accordance with some embodiments of the present inventive concept.

FIG. 1B illustrates a simplified schematic top view of the overall system for administering a substance to a subject in accordance with some embodiments of the present inventive concept.

FIG. 1C is a diagram of the system of FIG. 1B including chicks therein in accordance with some embodiments of the present inventive concept.

FIG. 2 is a diagram schematically illustrating movement of the subject and the possible errors associated therewith in accordance with some embodiments of the present inventive concept.

FIG. 3 is a diagram illustrating spatial variation in the x direction in accordance with some embodiments of the present inventive concept.

FIG. 4 is a diagram illustrating a result of not compensating for movement in the x-direction in accordance with some embodiments of the present inventive concept.

FIG. 5 is a diagram illustrating spatial variation in the y-direction in accordance with some embodiments of the present inventive concept.

FIG. 6 is a diagram illustrating spatial variation in the z-direction in accordance with some embodiments of the present inventive concept.

FIG. 7 is a diagram illustrating adaptive nozzle dosing in accordance with some embodiments of the present inventive concept.

FIG. 8 is a block diagram illustrating embodiments including multiple scanners positioned on the side of the belt in accordance with some embodiments of the present inventive concept.

FIGS. 9 and 10 are diagrams illustrating multibank nozzles in accordance with some embodiments of the present inventive concept.

FIGS. 11 and 12 are flowcharts illustrating processing steps in methods according to various embodiments of the present inventive concept.

FIG. 13 is a diagram comparing a whole chick scanning method and a slice method in accordance with some embodiments of the present inventive concept.

FIG. 14 is a diagram illustrating calculation of a position of the eyes of the bird as the bird gets close to the nozzle in accordance with some embodiments of the present inventive concept.

FIG. 15 is a block diagram of a system including a scanner and a data processor in accordance with some embodiments of the present inventive concept.

FIGS. 16A and 16B are diagrams illustrating partial pattern simultaneous dosing in accordance with some embodiments of the present inventive concept.

FIG. 17 is a high level diagram illustrating a generic subject that is delivered a substance in accordance with various embodiments of the present inventive concept.

FIG. 18 is a diagram of an injection system that may be used to deliver a substance in accordance with some embodiments of the present inventive concept.

FIGS. 19A through 19D are diagrams illustrating the use of methods discussed herein to deliver a substance to swine.

FIGS. 20A through 20E are diagrams illustrating the use of methods discussed herein to deliver a substance to a fish.

FIG. 21 is a diagram illustrating an example of training a machine learning model in accordance with some embodiments of the present inventive concept.

FIG. 22 is a diagram illustrating an example of applying a trained machine learning model to a new observation associated with identifying a location of the eyes of a bird in accordance with some embodiments of the present inventive concept.

### DETAILED DESCRIPTION OF EMBODIMENTS

The inventive concept now will be described more fully hereinafter with reference to the accompanying drawings, in which illustrative embodiments of the inventive concept are shown. This inventive concept may, however, be embodied in many different forms and should not be construed as limited to the embodiments set forth herein; rather, these embodiments are provided so that this disclosure will be thorough and complete, and will fully convey the scope of the inventive concept to those skilled in the art. Like numbers refer to like elements throughout. As used herein, the term “and/or” includes any and all combinations of one or more of the associated listed items. Similarly, as used herein, the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and this specification and will not be interpreted in an idealized or overly formal sense unless expressly so defined herein.

Reference will now be made in detail in various and alternative example embodiments and to the accompanying figures. Each example embodiment is provided by way of explanation, and not as a limitation. It will be apparent to those skilled in the art that modifications and variations can be made without departing from the scope or spirit of the disclosure and claims. For instance, features illustrated or described as part of one embodiment may be used in connection with another embodiment to yield a still further embodiment. Thus, it is intended that the present disclosure includes modifications and variations that come within the scope of the appended claims and their equivalents.

As discussed in the background, none of the conventional methods for delivering a substance, for example, a vaccine or other medicine, to a subject can adequately ensure that the correct dose of the substance was actually administered to the subject. Using the example of poultry, namely hatched chicks, one problem with automatic delivery of the substance to a chick is that chicks by nature move. Thus, when chicks approach the vaccination point in an automatic system, the chicks may be randomly oriented during “target acquisition” and, therefore, it is difficult to ensure that the chicks actually received the substance in the correct dose.

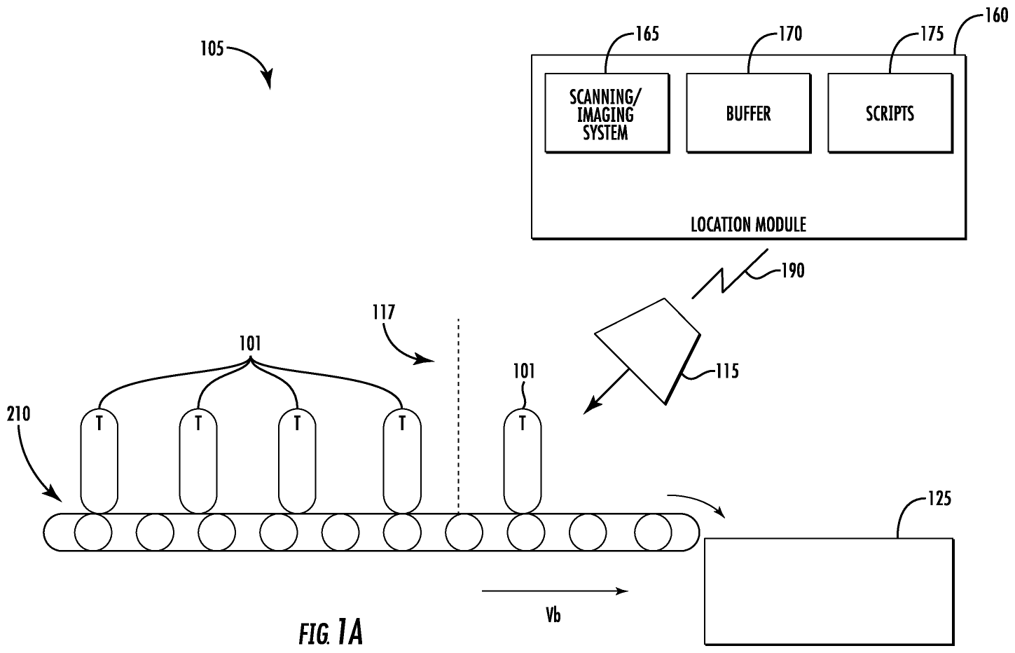

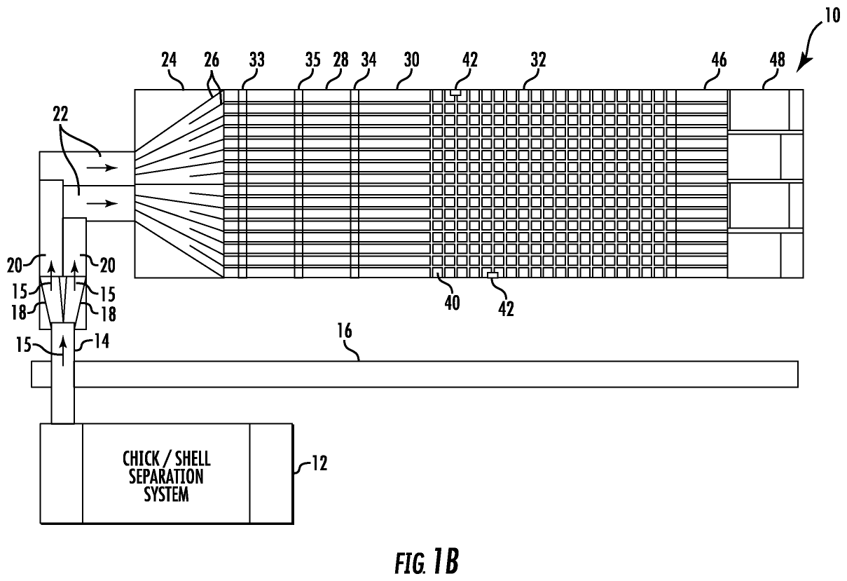

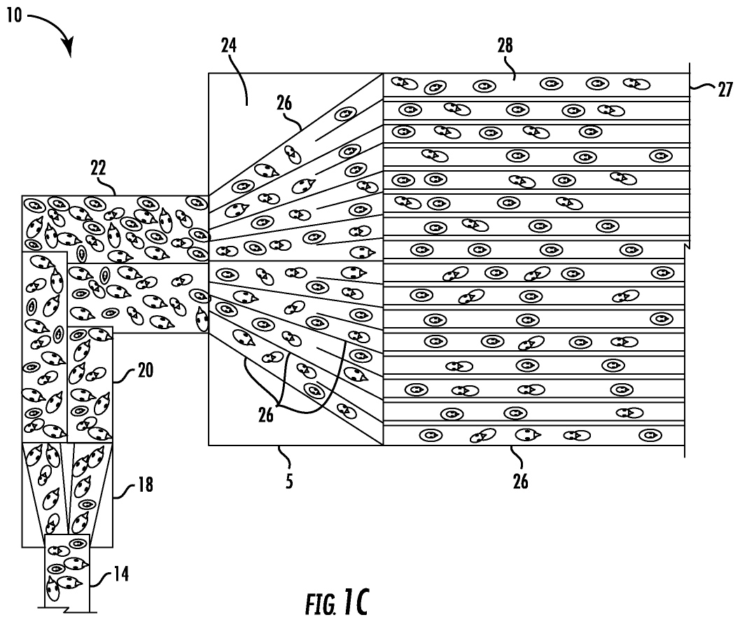

Furthermore, once the target is located (i.e. eyes on a chick), the chick can still move before administration of the substance, which also makes it difficult to ensure the chick actually received the substance in the proper dose. Accordingly, some embodiments of the present inventive concept provide methods for delivering a substance to a subject such that the method accommodates for variability in the position of the subject in three dimensions and minimizes a time between target acquisition, i.e. location of the subject, and delivery of the substance to the subject to increase the likelihood that the subject actually receive the substance in the proper dose as will be discussed further herein with respect to FIGS. 1 through 14.

As used herein, the term “subject” refers to the animal or human receiving the substance. Embodiments of the present inventive concept will be discussed herein with respect to the example subject of poultry, namely chicks. However, the subject may be any subject that could benefit from the methods, systems and computer program products discussed herein. For example, the subject may be any type of poultry including, but not limited to, chicken, turkey, duck, geese, quail, pheasant, guineas, guinea fowl, peafowl, partridge, pigeon, emu, ostrich, exotic birds, and the like. The subject may also be a non-poultry livestock, such as cows, ox, sheep, donkey, goat, llama, horses, and pigs (swine).

As further used herein, the “substance” refers to any substance that may be administered to the subject. For example, the substance may be a vaccine or other type of medicine. It is further contemplated that the substance may also be a topical coating or application of a solution that provides medicinal, cosmetic, or cosmeceutical benefit. For ease of discussion, embodiments discussed herein will refer to a vaccine. Furthermore, “target” refers to the location on the subject where the substance should be delivered. For example, using a chick as the subject, the target may be the eyes of the chick or any orifice of the chick or chicks face that may lead to the gut and/or respiratory tract of a chick. In some embodiments, the methods and systems discussed herein target each eye of the chick individually, which may create two distinct “target zones” per chick.

In particular, conventional methods and systems for administering a substance to a subject may not provide adequate assurance that the substance was actually received by the subject in the adequate doses. In the example of poultry, the substance, for example, vaccine, should be directed to the mucosa of a bird, for example, the mucosa in the eye(s) of the bird, the mucosa in an area around one or more eyes of the bird, the mucosa in nostrils of the bird, mucosa in a mouth of the bird, and/or mucosa in any orifice on a head of the bird that leads to the gut and/or respiratory tract. In some embodiments, the types of vaccines or other substances given to chicks by spray application to the mucosa may include, for example: vaccinations against Newcastle disease, infectious bronchitis virus, E coli, salmonella, coccidia, camplyobactor, Marek's disease, Infectious bursal disease, Tenosynovitis, Encephalomyelitis, Fowlpox, Chicken infectious anemia, Laryngotracheitis, Fowl cholera, Mycoplasma gallispticum, ND B1-B1, LaSota, DW, Hemorrhagic enteritis, SC, Erysipelas, Riemerella anatipestifer, Duck viral hepatitis, and Duck viral enteritis. However, as discussed above, embodiments discussed herein are not limited to poultry or birds. Thus, it is also anticipated that the embodiments herein may apply to the automated delivery of substance to the mucosa of other animals and mammals, including humans. In particular, there may be certain applications that may be appropriate for automated delivery of a substance to the facial mucosa of an infant or child, or disabled person. In addition, the automated delivery system described herein may have applicability to other animals, such as livestock, rodents and other animals raised commercially.

Referring now to FIG. 1A, a basic block diagram of a system 105 used for automatic delivery of a substance to one or more subjects will be discussed. As illustrated in FIG. 1, the system includes a conveyor belt 210 having a plurality of subjects 101 traveling thereon. As illustrated, the subjects 101 may be separated by an optional barrier 117 or placed in separate bins on the conveyor belt 210 in some embodiments. The system 105 further includes one or more spray nozzles 115 or nozzle banks in communication with a location module 160 in accordance with embodiments discussed herein. The nozzle 115 may communicate with the location module 160 using any communication method known to those having skill in the art. For example, communication 190 may be wired or wireless without departing from the scope of the present inventive concept.

The location module 160 communicates with the nozzle 115 such the nozzle knows when and where to deliver the spray including the substance to the target on the subject. As illustrated, each subject 101 includes a target area T illustrating where the substance should be delivered.

As further illustrated, the location module 160 includes a scanning/imaging system 165, a buffer 170 and a plurality of scripts 175 that are executed by a processor (1538 in FIG. 15). The location module 160 uses one or more scans of the subject 101 to determine a three dimensional (3D) position of the subject and directs the nozzle 115 to spray the substance at a particular time and location based on the determined 3D position. As used herein, the 3D position of the subject may be referred to as a 3D coordinate or 3D eye coordinate. The 3D coordinate is defined by the X, Y and Z positions of the subject/eye. In particular, the 3D coordinate (3D eye coordinate) discussed herein may consists of a scalar X position, Y position, and Z position, where any of these positions can be adjusted as needed to accurately define the 3D coordinate of the subject/eye. References to the X, Y and Z position, coordinates, directions etc. herein refer to the 3D coordinate of the subject/eye.

The scanning/imaging system 165 may include, for example, a two dimensional (2D) scanning system with a separate one dimensional (1D) sensor, a three dimensional (3D) scanning system or a 3D tomography system or any combination of 1D, 2D, or 3D sensors which are active or passive. Details of example methods of determining the X, Y and Z location of the subject 101 will be discussed further below. Once the substance is delivered to the subjects 101, the subjects 101 move down the conveyor belt 210 at a predetermined speed v_{b} and are delivered to a containment unit 125. It will be understood that the system 105 illustrated in FIG. 1A is provided for example only and, therefore, embodiments of the present inventive concept are not limited thereto. For example, although only a single nozzle and scanning system is shown, more than one of any element may be included without departing from the scope of the present inventive concept.

As used herein, a “scan” or “scanning” refer to scanning using systems incorporating a global shutter and/or a local shutter without departing from the scope of the present inventive concept. These scanning systems may be incorporated into an imaging system in some embodiments or may be a stand-alone system. Thus, it will be understood that any system that allows a user to obtain a scan or an image of the subject showing a location of a subject in accordance with embodiments discussed herein may be used without departing from the scope of the present inventive concept.

Embodiments of the present inventive concept will be discussed herein using a chick as the subject and the chick's eyes as the target of the sprayed substance. This has been done for ease of explanation and embodiments of the present inventive concept are not limited thereby.

As discussed above, a problem that occurs with automated spray delivery of a substance is that the subject, for example, a chick, moves. It can move up and down, side to side, forward and backward and any combination thereof. This causes a problem for the system 105 because the system 105 needs to know the position of the chick so that the substance can be properly delivered to the target, i.e. the mucosa of the chick's eye(s), the mucosa in an area around one or more eyes of the chick, the mucosa in nostrils of the chick, mucosa in a mouth of the chick, and/or mucosa in any orifice on a head of the chick that leads to the gut and/or respiratory track. Furthermore, once the chick's position is obtained/determined, the chick may move between position determination (target acquisition) and application of the substance, further complicating delivery.

An example system in which methods discussed herein may be used is illustrated in FIG. 1B. FIG. 1B illustrates a simplified schematic top view of the overall system for administering a substance to a subject in accordance with some embodiments of the present inventive concept. It will be understood that the simplified view does not include some of the equipment provided in various areas of the system 10. In embodiments where the subjects are chicks, the system 10 would likely be located in the day-of-hatch room in a chicken hatchery. As illustrated, the system 10 includes a chick/shell separator 12. The chick/shell separator 12 provides a means for separating the hatchling from its shell. A first conveyor 14 moves the chick from the chick/shell separator 12 through an opening in the separating wall 16 to a second, wider conveyor 18 in the direction of arrow 15. The separating wall 16 separates the shell separating process from the substance delivery process.

The second, wider conveyor 18 begins to spread the chicks out which makes processing each individual chick easier. From the second conveyor 18, the chicks are transported in the direction of arrows 15 onto third, and forth conveyors 20, 22 respectively, which are both wider than the conveyor 18. A fifth conveyor 24 has dividers 26 which may be suspended from the top of the conveyance assembly. The dividers 26 create lanes which help to move the chicks into narrow rows which eventually become single file rows. The chicks may travel on several conveyors (28, 30) through sensors (33, 34) and cameras 35 to a series of individual carrier devices 32 located below the angled conveyor belt 30. Each individual carrier device 32 is similar to a cup, cage or basket and sized to receive a single chick. The chicks may be sprayed 42 in the carrier devices 32 and travel on the conveyor 42 to the container 42. FIG. 1C shows system 10 having chicks therein. It will be understood that embodiments illustrated in FIGS. 1B and 1C are provided as examples only and embodiments of the present inventive concept are not limited thereto.

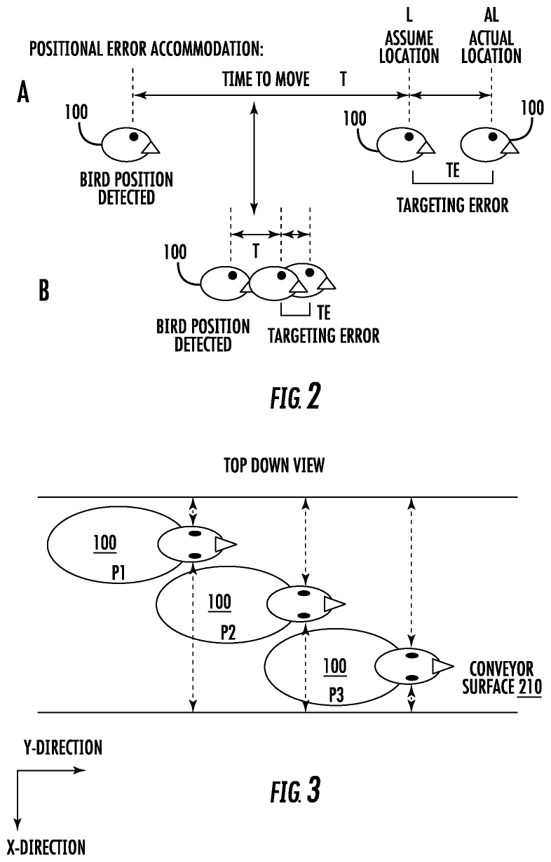

Referring now to FIG. 2, a diagram graphically illustrating the problems with movement of the subject discussed above will be discussed. As illustrated in FIG. 1A, the subject is a chick 100. The top line A illustrates the current situation with a chick 100, the amount of time the chick 100 has to move between target acquisition and application of the substance T, the assumed location at the time of application of the substance L, the actual location at the time of application of the substance AL and the error TE associated therewith. The second line B illustrates the same details in cooperation with methods and systems discussed herein, thus, decreasing the error TE as will be discussed further below.

In particular, as shown, in line A, the position of the chick 100 is determined and then while the chick 100 is waiting to receive the substance, it has a time to move T. Thus, the chick 100 may be assumed to be at location L. However, before the substance is actually administered, the chick 100 can move again and, therefore, the chick is not actually positioned at location L but at an actual location AL when the substance is delivered. Thus, there is an “error” TE associated with delivery based on the fact that the chick 100 is not where the system thinks it is when the substance is administered.

Accordingly, given that it is known that the chick 100 will move, up/down, back/front and side to side, embodiments of the present inventive concept take this movement into account when determining when and where to deliver the substance. In other words, in order to accommodate for a random orientation of the chick during acquisition, some embodiments of the present inventive concept determine the three-dimensional (3D) coordinates (X, Y, and Z) of the target area(s) (chick's individual eyes) and accommodate for the positional variances in the X, Y, and Z directions by varying delivery timing (e.g. the spray timing) for each individual eye on an individual chick basis. In order for the 3D positional (X, Y, and Z) information to be useful, a response time between determining the position and administration of the substance should be reduced as much as possible or minimized.

Referring again to FIG. 2, in line B, the time T between determining the position of the chick 100 and actually administering the substance is drastically reduced. Thus, the targeting error TE may also be reduced. Reducing the amount of time between determining the position (scan) of the chick 100 and the time the substance (spray) is delivered to the target area of the chick 100 may subsequently reduce the chance of error in the delivery system. Thus, in accordance with some embodiments discussed herein, as the overall system response time goes down, the amount of time the chick has to move between scanning for position and delivery (spray) is also reduced. This reduces the average amount of targeting error (TE) associated with chick movement.

As will be discussed further herein, some embodiments of the present inventive concept provide methods, systems and computer program products for adjusting for positional changes in the subject to provide accurate delivery of a substance (spray) to a target zone of the subject (chick eyes). Furthermore, some embodiments provide strategies for improving the effectiveness of the delivered dose and decreasing a time from scanning to delivery.

To adequately accommodate for movement by the subject in all three directions, X, Y and Z, errors for each must be considered and computed as will be discussed below. In particular, some embodiments of the present inventive concept provide “adaptive nozzle timing.” “Adaptative nozzle timing” refers to the ability of the spray system to individually assess a 3D position of each chick/subject and individually change the timing of delivery (spray timing) for each delivered dose. In other words, each chick's 3D coordinates are determined and used to choose the timing of the delivery to increase the likelihood that the substance hits the target (eyes) and that an adequate dose is delivered.

Adaptive nozzle timing takes X, Y, and Z directions into account in accordance with example embodiments discussed herein. Referring first to FIG. 3, a diagram illustrating X-direction spatial variation in accordance with some embodiments of the present inventive concept will be discussed. The X-direction adaptive nozzle timing in accordance with embodiments discussed herein accommodates for the position of the chick across a width of the conveyor belt 210. The chick 100 is traveling on a conveyor belt 210 towards a delivery system that will deliver the substance to the chick 100. As illustrated in FIG. 3, the position of the chick 100 may vary in the X-direction on the conveyor belt 210. In particular, the chick 100 in position P1 is moving down a left side of the belt 210, the chick 100 in position P2 is moving down a middle of the belt 120 and the chick 100 in position P3 is moving along a right side of the belt 210. It will be understood that there is only one chick on this portion of the belt at time of delivery, however, FIG. 3 illustrates a same chick in three different positions on the belt 210 for example purposes. It will be understood that although only three positions are illustrated any number of positions may be accommodated for without departing from the scope of the present inventive concept. If it is assumed that the chick 100 is always traveling down the center of the belt 210, i.e. in position P2, then delivery will be off target in positions P1 and P3.

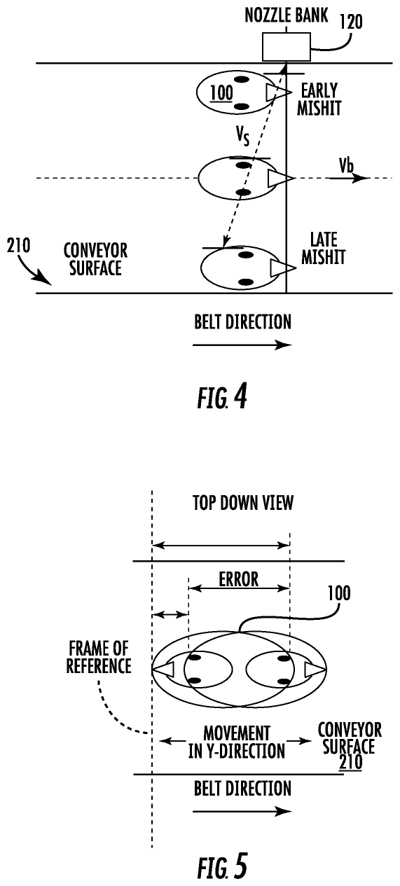

The effect of not accommodating for each position P1, P2 and P3 is illustrated, for example, in FIG. 4. As illustrated therein, the chick 100 is traveling on the conveyor belt 210 at a velocity v_{b} towards the nozzle bank 120. As shown, a chick 100 in position P1 would not receive the spray in the eyes (early mishit) and the chick 100 in position P3 would be too far down the conveyor 210 and also would not receive the spray in the eyes (late mishit). Thus, in accordance with some embodiments of the present inventive concept, two main accommodations are made in the timing of the spray calculations in order to accurately target in the x-direction. These two accommodations are the velocity of the spray (vₛ) as it travels through the air and a distance (d) from each sprayer to the target zone for each chick.

In particular, the nozzle 120 sprays the substance in a vector with a known velocity (vₛ) such that the target area, for example, the eyes of the chick 100, moving on the conveyor 210 at a velocity v_{b} intersects directly under the spray pattern at the precise instant the fluid pattern comes in contact with the target area. The distance the chick travels along the belt before the target area intersects the spray pattern is a function of the velocity of the spray vₛ, the velocity of the target vₜ (chick) moving along the conveyor, and the distance (d) from the spray nozzle to the target area. Thus, a useful relationship is defined as follows:

$$( Time of Flight ) = ( Distance from Nozzle to Target ( {d}_{tn} ) ) / ( Speed of Dispensed Fluid ( {v}_{s} ) ) \qquad Eqn . ( 1 )$$

where Time of Flight (TofF) is the time the chick 100 travels on the belt 210 while the spray is in transit to being delivered; the distance d is the distance from the spay nozzle to the target area and the speed vₛ is the speed of the spray from the nozzle. The spray timing for each chick 100 is individually calculated based on the X-location of the chick's eyes (target) with respect to a width (w_{c}) of the belt 210. As discussed above, if this dimension is not accounted for it would result in the spray pattern reaching the chick's eye more quickly when the chick is closer to the nozzle (early mishit-FIG. 4) and would result in the spray pattern reaching the chick's eye late when the chick is farther away from the nozzle (late mishit-FIG. 4). The adaptive nozzle timing in the x-direction is calculated for both the right and left eyes independently creating two distinct “target regions” with their own X positional accommodations and calculated nozzle timings.

The distance (dₜₙ) from the nozzle 120 to the target (chick's eyes) can be determined as follows: Assuming the conveyor belt has a width (w_{c}) of 6 inches, that the chick is positioned in the center of the belt (½ the width of the belt at 3 inches), that the chick's eyes are the target and a 1.0 inch width of the chick's head, the chick's eye (target) may be 2½ inches from the nozzle if the chick is looking forward. This is a 6 inches belt width minus half the width of the belt (3 inches) and half the width of the chick's head (0.5 inches.)

It will be understood that compensating for the fluid spray velocity (vₛ) is only a first step in accommodating the positional variabilities in the X-direction. If embodiments of the present inventive concept only compensated for the spray velocity, the system would be accurate only when the chick's head was directly in line with the centerline of the belt 210 placing the eyes evenly about the centerline, but would target with a progressively greater amount of error the larger the distance from the centerline. By taking into account both the fluid velocity spray timing offset as well as the x-position along the belt with respect to the spray nozzles, a precise spray timing accommodation can be achieved for accurate fluid delivery to the target zone.

A sample calculation of Adaptive Nozzle Timing in the X-direction is set out below. In the following example, belt speed (v_{b}) is assumed to be 30 inches/second (in/s); the spray velocity (vₛ) is assumed to be 200 in/sec; a width (w_{c}) of the conveyor belt is assumed to be 6 inches (in.) and a width of the chick's head (w_{bh}) is assumed to be 1.0 in. Using Eqn. (1) set out above (TofF=(w_{c}−dₜₙ)/vₛ):

$$TofF = ( 6 in . / 2 ) - 1 / 2 in . ) ) / 200 in / sec . = 0.0125 s$$

Thus, TofF for the chick 100 is 0.0125 s. The error can be calculated as follows:

$${D}_{error} = {v}_{b} * TofF \qquad Eqn . ( 2 )$$

where the Dₑᵣᵣₒᵣ is the distance error; v_{b} is the speed of the belt and TofF is the calculated time of flight, which yields:

$${D}_{error} = 30 in / s * 0.0125 s = \text{.375} inches$$

Thus, the system should correct the positioning of the nozzle by 0.375 inches in the X direction. It will be understood that this is provided as an example only and that other widths, speeds etc. may be used without departing from the scope of the present inventive concept.

Although embodiments of the present inventive concept provide examples where the substance is provided in a straight line across the belt on which the chicks are traveling. It will be understood that embodiments of the present inventive concept are not limited to straight sprays. For example, the substance may be sprayed at an angle relative to the belt without departing from the scope of the present inventive concept. In these embodiments, the nozzle(s) may be positioned to produce the spray at the desired angle.

As discussed above, embodiments of the present inventive concept adjust for X, Y and Z directions. Adaptive Nozzle Timing for the Y-direction will now be discussed. The adaptive nozzle timing accommodates for positional variance of the targeting area (chick's eyes) along the length of the belt. Similar to the X-direction compensation the Y-direction compensation measures a position of the target area along the Y-axis of the belt (the length of the belt) and adaptively varies the spray timing for each chick to cause the spray pattern to intersect the eyes even for varied target positions along the Y-axis. As illustrated in FIG. 5, the chick 100 can move forward and backward along the belt 210. If embodiments of the present inventive concept did not accommodate for the Y-dimension, for example, using a set time value, the targeting would only be accurate for a single point on the belt and a large source of error would be induced causing inaccuracies in the targeting. The adaptive nozzle timing in the y-direction is calculated for both the right and left eyes independently creating two distinct target regions with their own Y positional accommodations and calculated nozzle timings.

The equation defining spray timing is a direct measurement of the Y-positioning of the chick's eyes along the direction of the belt 210 and adaptively accounting for the varying delays required to turn the sprayer on to cause the spray pattern center to intersect with the target zone for each individual chick. The amount of time to delay spraying can be calculated using Eqn. (4) below.

$${Delay}_{Spray} = ( {d}_{m} ) / ( {v}_{b} ) \qquad Eqn . ( 3 )$$

where Delay_{spray} is the amount of time the system should delay spraying the chick; dm is the measured distance is a y-coordinate for the target, e.g. an eye(s) of the chick and the v_{b} is the speed of the belt.

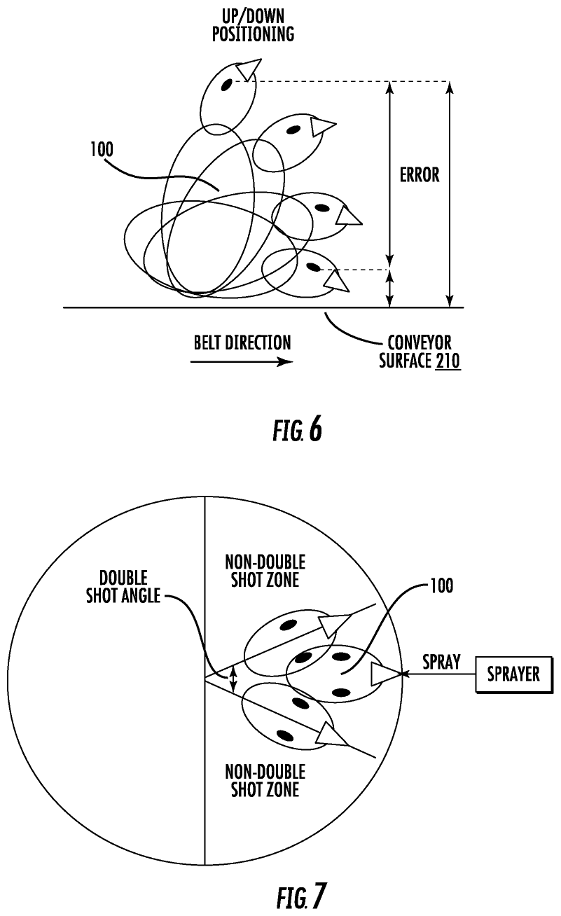

Similarly, FIG. 6 illustrates the chick's movement in the Z-direction (distance from the belt), up and down as shown. Thus, the “error” illustrated in FIG. 6 is the displacement of the chick 110 up and down perpendicular to the conveyor belt 210. Accommodation in the Z-direction is achieved by accurately measuring a position of the target area (chick's eyes) in the Z-direction and selecting a “spray pattern” for an array of height selections (delta up and down) such that the sprayed pattern is centered around a height of the chick 100 as shown in FIG. 6. This could also be calculated for both the right and left eyes independently creating two distinct target regions with their own Z positional accommodations.

Embodiments of the present inventive concept discussed above adjust a position of a nozzle delivering a substance to a subject, for example, spraying a vaccine on a chick or piglet, and adjust the timing of the spray to accommodate for movement of the chick or piglet in the X, Y and Z positions. However, in some embodiments, movement in the X, Y and Z directions may be accommodated by providing a bank of nozzles that moves to the position of each chick. For example, this may be a manifold of orifices which each shoot a stream of liquid or a manifold of spray cones. In some embodiments, the manifold may be placed on a gantry which can move in the X, Y, and Z planes. By doing so, the manifold would spray the same nozzles for each chick, piglet, or fish but the position of the manifold would be adaptively moved to accommodate for the height of the target zone, distance along the length of the belt, and the timing of the spray would be adaptively varied to accommodate varying target zone positions along the width along the belt. Furthermore, in some embodiments, the nozzle banks could be moved to a position that is as close to the scanning as possible. This can include adaptively moving the nozzle bank(s) on an individualized chick, piglet, or fish basis to minimize the time from imaging to spray by placing the nozzles as close as possible for each subject regardless of orientation.

The goal of a spray system is to deliver a defined dose to the target area of the chick (the eyes). Because the position of the chick's eyes is dependent on the orientation it holds its head during the scanning and spray cycle there are certain orientations where one of the sprayers may not see the target area, i.e. one or both eyes. The various positions of the chick 100 are illustrated, for example, in FIG. 7. In these embodiments, it may be beneficial to adaptively vary the nozzle dosing, such that the sprayer which can see the target area effectively delivers a dose to one or both eyes from a same sprayer. One notable orientation where this is of benefit is spray manifolds oriented 180 degrees opposed from each other. In this situation if a chick has both eyes looking directly at a single spray nozzle, the back of its head would be pointed at the opposing nozzle. Instead of firing one nozzle at the eyes and the other at the back of the head, embodiments of the present inventive concept recognize that the chicks head is facing away from the one nozzle and, therefore, would deliver a full dose for each eye to be delivered from the nozzle band that the chick is facing and not spraying anything from the opposing nozzle. Furthermore, a “double shot” angle may also be defined where if a chick's head is oriented within a specified number of degrees from looking directly at one of the spray nozzles this “double shot” function is activated, and the sprayer adaptively changes to targeting both eyes from a single bank. In embodiments including an upstream and downstream nozzle bank, the decision can be made to fire on one eye from the upstream bank and the second eye from the downstream bank. This configuration may allow for optimization in both spray angles and spray timings. It will be understood that embodiments including the adaptive sprayers may also accommodate for the changes of position in the X, Y and Z positions as discussed above and may be used to target either one or both eyes. Referring again to FIG. 7, the “double shot angle” may be considered the optimum angle for firing the spray from a single side such that the percentage of eye/face hits on a chick is maximized based on the chick's anatomy.

In some conditions it may be beneficial for the system to target only a single eye. For example, targeting a single eye may reduce dispense volume or allow firing of all vaccine particles into one eye. In embodiments using chicks or birds, the angle of the head can be used in order to determine the optimum eye to spray. The eye which is most orthogonal to the spray heads can be chosen in order to provide the most direct hit. Furthermore, if the angle between right and left spray nozzles is equivalent then the eye closest to the spray nozzle can be chosen in order to reduce, or possibly minimize, the time of flight and thereby minimize the time from imaging to spray.

One disadvantage to positional scanning as discussed above is that the scanning is acquired from a top down view. Thus, during the scan, they eyes of the chick are not directly scanned. Because the eyes, in some embodiments, are the spray target area, the position of the eyes is computed based on anatomical assumptions of the chick. Thus, some positions of the chick's head are not accommodated for where the assumed anatomical offsets are not correct. For example, in some embodiments, a height of the chick is found, a predetermined geometry is fit to a subset of the data, then an assumed position for the eyes (target region) is calculated. If the chick were to rotate their head such that they were looking straight up, straight down, or were to cock their head to the side, there would be no way of knowing that the assumed anatomical positions of the chick were in fact incorrect. In some embodiments, this is addressed by directly scanning the eyes. For example, as illustrated in FIG. 8, scanners 450 and 451 are placed at an angle to the conveyor 210 such that they have the ability to directly scan the eyes. Embodiments illustrated in FIG. 8 provide the benefit of eliminating the positional error caused by inaccurate anatomical assumptions.

Using “Direct Eye Imaging” illustrated, for example, in FIG. 8, an image processing algorithm operates on the image data to compute the location of the target regions (e.g., eyes). A simple example of such an algorithm would exploit the fact that the eyes are among the darkest parts of the image by selecting all pixels that are darker than a threshold brightness value, group adjacent selected pixels, and calculate the center location of the group as the eye location. The algorithmic computation for determining the position of the eyes could use a simple thresholding algorithm to increase the contrast between the eyes and the feathers causing the eye to stand out and be easily detected. Very little spatial resolution is needed to run an algorithm to perform this function and a scanner less than 1.0 MP is sufficient for eye detection. This allows for an improvement in overall system response time, improving targeting performance. Infrared including near infrared (NIR), short-wave infrared (SWIR), mid-wave infrared (MWIR), or long-wave infrared (LWIR), visible light spectrum, Ultraviolet (including the “UVA”, “UVB”, and “UVC” bands), or other wavelength devices (imaging devices and/or illumination sources) may be used to improve detection of bird anatomical features (e.g., feathers, eyes, beak, nostrils, etc.)

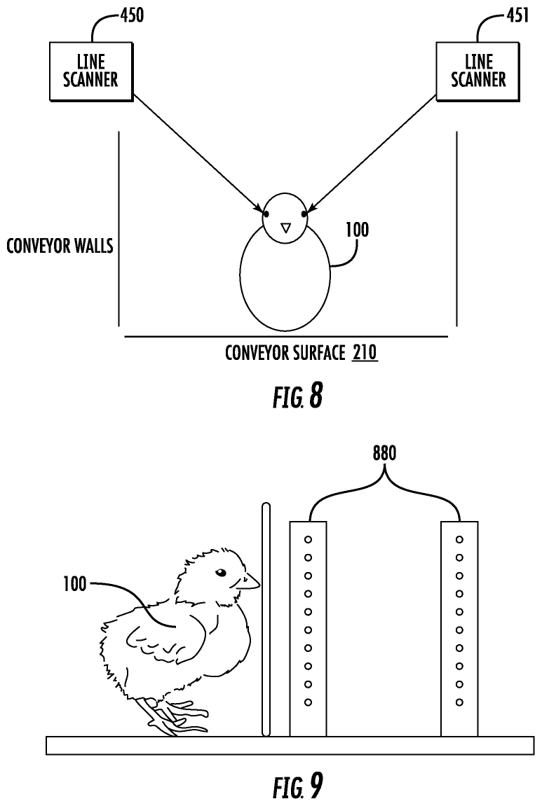

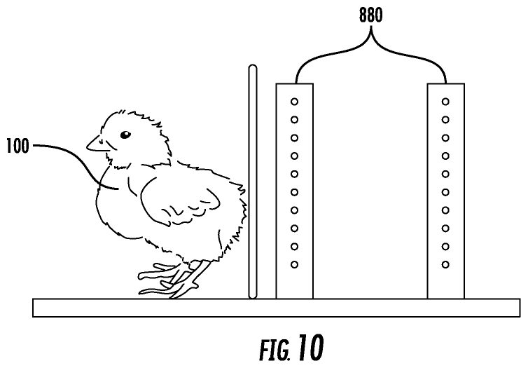

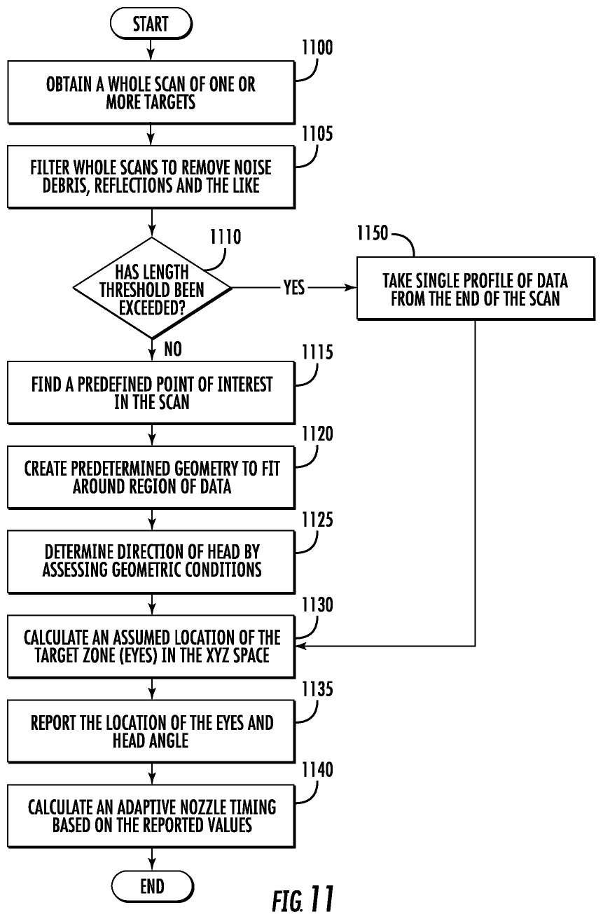

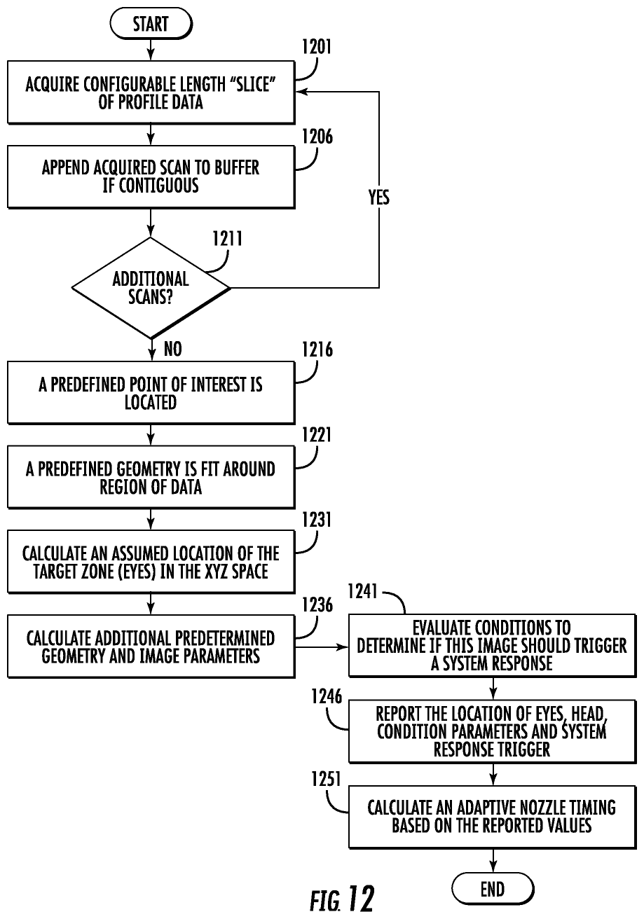

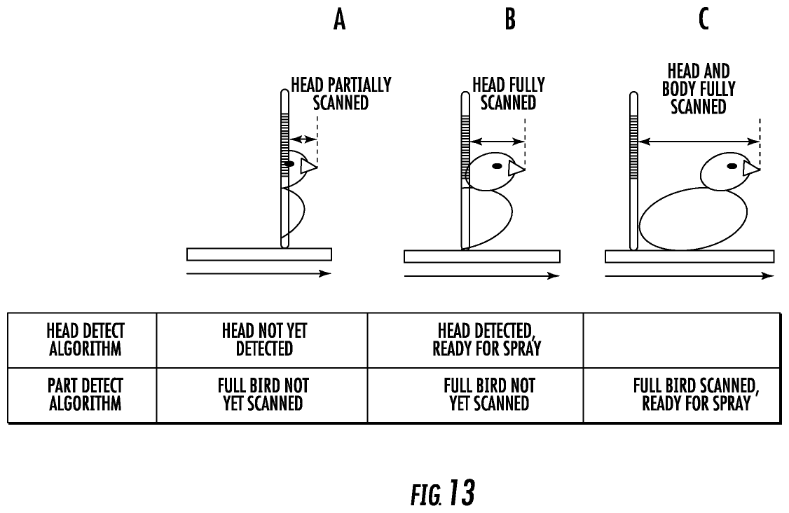

As discussed above, some embodiments of the present inventive concept may include multibank nozzles 880 illustrated, for example, in FIGS. 9 and 10. By having multiple (multibank) nozzles positioned at different distances from the scanning system, the time from scanning to spray can be reduced, or possibly minimized. See e.g., FIG. 2. For chicks in the forward orientation (FIG. 9) the head is farther down the belt than the body, for chicks in the rearward orientation (FIG. 10) it is the opposite. By having multiple nozzle banks positioned at different locations along the belt, a nozzle can be prepositioned to minimize the system response time for chicks of various orientations. A forward facing chick (FIG. 9) can be sprayed by the far bank and a rearward facing chick (FIG. 10) can be sprayed by the near bank. The position of the nozzles can be optimized such that average system response time is minimized. For example, this may be done by measuring the distribution of chick body positions as they travel down the belt and using the statistical likelihood to orient themselves in a particular posture. This data set can be bolstered in real time in the system to adaptively learn the positions chicks are most likely to orient themselves in. This data may then be used to set the optimum nozzle positions that will, on average, reduce, or possibly minimize, the response time between imaging and spray. As discussed above, in some embodiments, instead of spray manifolds in fixed positions the manifold can move to each chick after it is scanned in order to minimize the wait time from imaging to scan.

Details with respect to scan acquisition using, for example, three-dimensional (3D) scanning or 3D tomography discussed above will be discussed. Point clouds, containing an array of pixels with additional displacement, color, and/or intensity information are generated by, for example, a scan (a row of pixels) or area scan (an array of pixels) device. The device may be one or more devices and can scan from directly above a target, from either side of the target, or any other position without departing from embodiments discussed herein. The generated scans may be analyzed as one or separately, for example, stereovision, to create “images.” The scanning device may have internal or external trigger mechanisms and may or may not buffer or continuously stream scans or pixel information.

In particular, an “LMI” (e.g. Gocator brand) is a scan laser profilometer that reports profiles, or single rows consisting of data points with X, Y, and Z (displacement, or height), and intensity information. The device may be used in a continuous “free-run” mode. In this mode, the device continuously takes profiles, and has an on-board algorithm that buffers each profile and uses a programmable threshold to begin and end the image. A two-dimensional (2D) array of XY coordinates with the additional Z height and intensity information is passed to the analysis algorithms (analysis module). It will be understood that the “free-run” mode algorithm is a known algorithm, a core feature of the sensor, from the sensor manufacturer. Other algorithms may be used without departing from the scope of present inventive concept.

The location module performs an image analysis to take a whole (or partial) scan (or point cloud) of a target (chick) and report an inferred or directly measured XY position of the target zone (for example, the eyes of the chick in case of the chick) (or also including Z coordinate). The Z height may be measured from the scan indirectly or may be directly measured without departing from the scope of the present inventive concept.

Referring now to the flowchart of FIG. 11, processing steps for whole scan analysis will now be discussed. As illustrated in FIG. 11, processing steps begin at block 1100 by returning a “whole scan” of the target, for example, single or multiple chicks. Once the scan or scans are obtained, the scan or scans are analyzed wholly or partially to determine a location of a target zone(s), for example, the chick's eyes, in the scan. As discussed above, the “target” or “target zone(s)” is the location on the target for delivery of the substance, for example, the vaccine. For a chick target, one or both eyes of the chick would be the target zone(s). When the target is a chick, this analysis may look for a head or other distinguishable feature of the chick and may report the inferred left and right eye positions. The directly measured eye position Z values or the “peak” value used to find the head may also be reported. Further details with respect to directly detecting eye position versus inferring eye position will be discussed further below.

When using LMI, the onboard algorithm on the LMI processes each whole scan reported by the “Part Detect” algorithm including therein. Operations proceed to block 1105 where the obtained whole scan is filtered to remove any noise caused by debris, reflections, and the like.

The whole scan is analyzed and it is determined if it conforms to a set or subset of geometric conditions and calculations. Now, specific system responses can occur. The image is assumed or determined to contain the region of interest, and the XYZ algorithm as described then executes (block 1115).

If it is determined that scan length has not been exceeded (block 1110), a predefined point of interest in the scan is found (block 1115). A specifically defined region of data around the point of interest is taken, and a predetermined geometry is fitted around the data in this region appropriate to the subject type being measured (block 1120). The direction of the chick's head is determined by assessing geometric conditions in light of the known anatomical structure of the chick's head. (block 1125) An assumed location of the target zone (eyes) in the X, Y and Z space is calculated (block 1130).

The algorithm module may use a custom script written in an interface and language provided by the manufacturer of the sensor (for example, C). The custom script may define an offset in millimeters (mm) corresponding to the assumed eye position “Forward” and “Sideways” (from the predetermined geometry's center point). Once determined (block 1130), the location of the eyes (eye positions) and head angle are reported (block 1135). An adaptive nozzle timing based on the reported values is calculated (block 1140).

If the calculated overall length of the scan is above a predefined threshold (block 1110), the target (chick) is assumed to have moved during acquisition of the scan. In these embodiments, the single profile of data from the end of the scan is used (block 1150). Operations proceed directly to block 1130, bypassing the other measurements and calculations.

In some embodiments discussed herein, algorithms are built using sensor manufacturer provided tools, organized into a toolset with inputs, outputs, and data flows between the tools, feeding into the custom-written Script portion of the algorithm. However, it will be understood that embodiments of the present inventive concept are not limited thereto.

Referring now to FIG. 12, a flowchart illustrating processing steps in methods detecting a head/eyes of the chick in accordance with some embodiments of the present inventive concept will be discussed. In these embodiments, unlike in embodiments discussed above with respect to FIG. 12, the sensor no longer returns a single scan of the single chick/target. A slice, of the chick is imaged, then added to a buffer such that for every image with predefined slice length the image of the chick is added to, until the entire chick is imaged. Each time the buffer receives a new “slice”, the scan is analyzed. In particular, operations begin at block 1201 by acquiring a slice of data from the target/chick. The acquired slice is provided to a buffer and appended to the already buffered slices, if any (block 1206). It is then determined if additional scans are needed to obtain additional slices to obtain a scan of the entire target/chick (block 1211). If additional scans are needed (block 1211), operations return to block 1201 to obtain a new slice. If, on the other hand, it is determined that no additional scans are needed (block 1211), operations proceed to block 1216.

An overall length of the scan is calculated and it is determined if the length exceeds a predetermined threshold. If the threshold has been exceeded, a new scan is returned for processing every pre-defined increment of travel (i.e. the length of a “slice”). If the threshold is not exceeded, the scan is returned for processing. The processing module contains a special tool designed to buffer the defined length “slices” discussed above. Each time a scan is returned, if there are more than a configurable number of scans already in the buffer, the buffer is cleared. Each scan also knows “where” the last profile was taken along the direction of travel. If the last returned image is further than the defined slice length from the previous profile in the buffer, in other words not a contiguous image, the buffer is cleared.

Due to the scan nature of the sensor, a full image of a chick is acquired one slice at a time and passes beneath the sensor as it builds the single profiles into a full scan of the target. The system response time includes the time it takes for the entire chick to pass underneath the laser line before a scan can be analyzed, as well as the additional analysis time. In these embodiments, each partial scan (slice or slices combined) is analyzed simultaneously with the next “slice” being acquired, and as seen in FIG. 13, a partial scan containing a head can trigger a system response (spray from nozzle), saving the additional acquisition time of the remaining portion of the image that does not contain a head. In other words, using slices, only the head with the chick's eyes needs to be acquired before the chick can be sprayed. Thus, time can be reduced by the time it would take to scan the remaining portion of the chick. This graphically illustrated in FIG. 13.

In particular, as illustrated in FIG. 13, in frame A, only a portion of the chick's head has been scanned using both methods discussed above, i.e. whole chick and slices. However, in frame B, the entire head of the chick has been scanned, but the completed chick is not scanned until frame B. Thus, using the slice method, a spray may be performed after frame B since the target (one or more of the chicks' eyes) have been scanned and their location known. Thus, the slice method may be used to reduce the timing between detection and spray as it does not have to wait for the scanning of the whole chick.

In some embodiments, the bird/chick may be tracked through multiple frames with an algorithm to allow the bird to approach the nozzles as closely as possible before positions are locked in and timing adjustments are made to spray pattern. A simple example of such an algorithm would detect the target area (an eye in this example) in frame B of FIG. 13 using, for example, steps of a method discussed above with respect to FIG. 8 and then predict the location of the eye in frame C using the known speed of the bird/chick motion. Then, using the same detection method in frame C, the algorithm may identify the detected dark pixel group nearest the predicted location as the same eye that was detected in frame B. This process may continue through subsequent frames until the location predicted from a current frame to an upcoming frame has progressed beyond the point that it can be sprayed. At that point the eye location from the current frame is used to lock in the spray pattern.

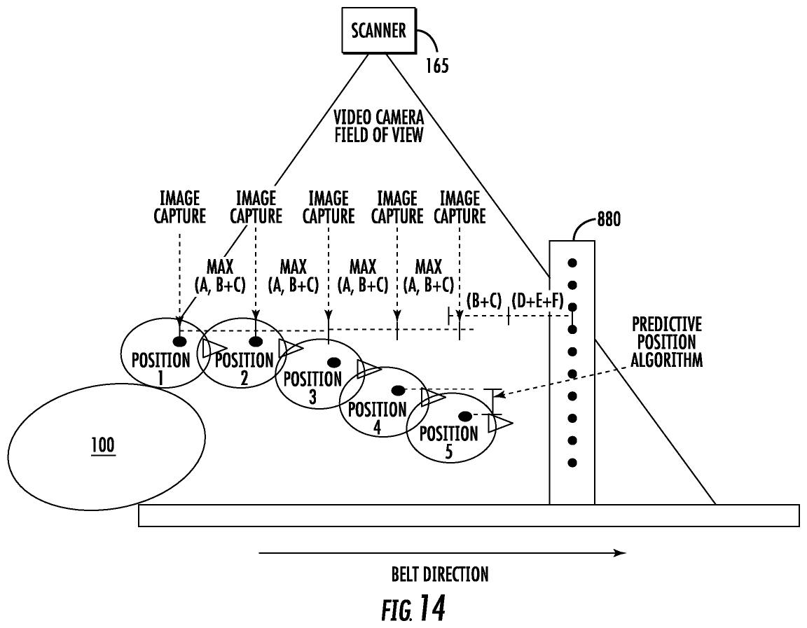

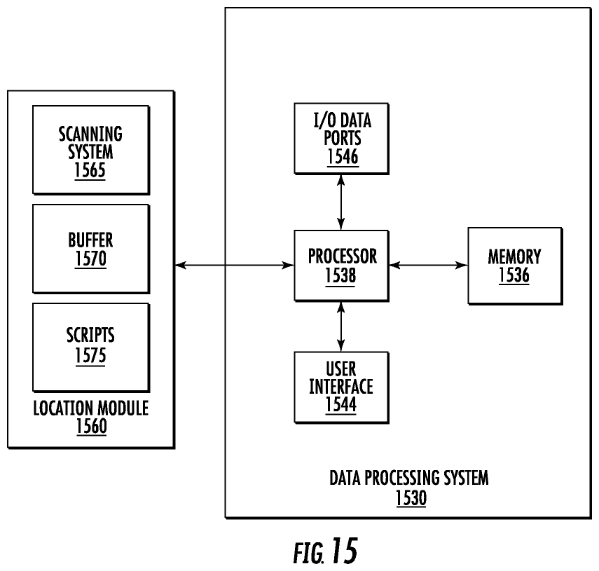

Referring to FIG. 14, embodiments of the present inventive concept for progressively scanning allowing the subject/bird to approach the nozzles as closely as possible will be discussed. As illustrated in FIG. 14, target zone tracking may allow the positional information to be as fresh as possible, reducing time from imaging to spray. This would also open the possibility for simplifying the system without any loss in performance by using a single set of spray manifolds as opposed to the dual set shown in FIG. 10, 880 without any loss in system response time. This would be a significant benefit in both reducing system complexity, system maintenance costs, and overall system hardware costs. In particular, by progressively scanning for the target zone as shown in FIG. 14 (position 1, position 2, position 3 . . . position n) and calculating the subject's position as it moves along the belt, a predictive positional algorithm can also be applied to modify the final assumed position of the target zone. This type of algorithm predictively accommodates the movement of a bird during the time from imaging to spray by utilizing the direction of movement the bird was in moments before its final position was locked in. It then uses that velocity and acceleration rate to predict a final location of the bird at the exact moment of impact of the spray. Artificial intelligence/machine learning discussed below may also be used along with algorithms for kinematic consistency in order to improve the position estimate of the eyes. Tracking eye pair positions and orientations in 3D can be used to reduce false alarms by feeding the tracking algorithm anatomical data to bound what it is looking for. For example, a chick eye pair on a given bird should be within a certain distance from each other.

Referring again to FIG. 12, operations proceed to block 1216 where the obtained scan (all slices together) is filtered, then a predefined point of interest is found. A predefined geometry, appropriate to the subject area being measured, is fit around the predefined point of interest (block 1221). The direction of the chick's head is determined by assessing the target region in light of the known anatomical structure of the chick's head.

An assumed location of the target zone (eyes) in the X, Y and Z space is calculated (block 1231). Additional predetermined geometry and image parameters may be calculated to provide further and more refined positional information, for example, more refined alignment of the head in space (block 1236). As discussed above, a custom script is then run on the sensor. Eye offsets are defined, head direction found, and eye positions inferred. However, in embodiments illustrated in FIG. 12, all these characteristics are fed into a rubric of conditions designed to determine if the current scan is valid, i.e. should this scan trigger a system response (block 1241). Conditions may include, for example, the predetermined geometry is not fit to the chick in a manner characteristic of a chick's head, the inferred eye points are too close to the end of the scan or do not match anatomical assumptions, the last line of the image is not characteristic of a “complete” image, so on and so forth. Some conditions are taken in combination, or independently. The custom script then reports this evaluated condition (true or false) to the control system, along with the eye positions, heights, and the like (block 1246). A bit field for parsing these conditions and other information is also returned. The control system evaluates the true false condition to determine if a response should be taken to the reported XYZ eye coordinate information, or if it should wait for the next image to be processed. Once it is determined that the current image should be used, an adaptive nozzle timing based on the reported values is calculated (block 1251). The adaptive timing is used to spray the target at the appropriate time to increase the likelihood of a successful spray.

As discussed above, some embodiments of the present inventive concept infer a position of the eyes of the bird and use this inferred position as an input to the algorithm. It will be understood that not directly imaging the eyes of the bird to determine their position may impose problems in the system. For example, as illustrated in FIG. 14, when a bird 100 is imaged/scanned 165 from the top down the eyes cannot be directly seen, therefore, the position of the eyes must be algorithmically computed. This leaves room for corner cases where the computed locations of the eyes have very low accuracy. Directly imaging the eyes may reduce, or possibly, eliminate this failure mode. Furthermore, the time from imaging to spray can be drastically reduced if the eyes are directly imaged. This is because the eye, which is the “area of interest” and is the target zone in some embodiments, can be tracked as the bird moves down the conveyor belt and the locking in of the position of the eyes can be delayed until the eyes are very close to the sprayer 880. This allows the system to constantly compute the eye positions for birds within the field of view as they approach closer and closer to the spray nozzles 880. Once the birds are very close to the spray nozzles the positions of each eye can be independently locked in allowing very little time for bird movement. Furthermore, the difference in positions can be used to measure the speed at which a bird may be moving and adaptively predict the position the “target zone” (i.e. eyes) will be in at the moment the spray impacts the bird.

Various variables may be relevant when performing direct eye imaging. These include a frame period, exposure time, algorithm processing and communications, valve response time, flight time and dose time. It will be understood that other variable may also be relevant without departing from the scope of the present inventive concept.

As used herein for the purposes of discussion of, for example, FIG. 14, “frame period” refers to an amount of time between image acquisitions for a video camera of a given frame rate (expressed in frames per second (fps). “Exposure time” refers to an amount of time a digital sensor is exposed to light for each frame taken by the video camera. That amount of time is the shutter speed and is expressed in fractions of a second. A 1.0 ms shutter would be 1/1000th of a second shutter speed. “Algorithm Processing and Communication” refers to the amount of time required to analyze and process the image acquired by the digital camera and determine the X, Y, and Z coordinates of each eye of the bird. “Valve Response Time” refers to the amount of time required for the electromechanical valve controlling the spray to open. “Flight time” refers to the amount of time for the liquid coming out of the nozzle to traverse through the air and impact the target. “Dose Time” refers to the amount of time the valve is left in the open position. This along with the belt speed defines the length of the pattern applied to the target.

As illustrated in FIG. 14, as the birds eye traverses along the belt into position 1, the bird 100 enters into the field of view of the video camera 165. The video camera scans every frame for the eye looking to compute its position. The camera sees the eye for the first time when the bird enters position 1 and computes its 3D coordinates. During this computation time the bird has moved to Position 2. Based on the remaining distance to the spray nozzle and the necessary timing compensations to fire at a bird with the given X, Y and Z coordinates, the system will decide if an additional X, Y and Z position can be computed allowing the bird to get closer to the nozzle before the positions are locked in. FIG. 14 illustrates that the system calculates new X, Y, and Z coordinates at Positions 2, 3, and 4. The bird translates along the belt while its eye positions are computed and by the time the X, Y and Z coordinates from Position 4 are computed, the bird's eye has moved to position 5. At this point the bird's eye is getting very close to the spray nozzles 880. The eye of the bird 100 at Position 5 cannot be computed because if this was done the amount of time required to return the X, Y and Z coordinates would cause the eye to translate too near to the nozzle 880 to allow the valve response time, flight time of the spray, and dose of the spray to be adequately compensated for. Therefore the X, Y and Z coordinates for Position 4 would be used for this bird as it is the closest possible X, Y and Z coordinates to the spray that the system could capture. Note that the birds orientation changed from Positions 1 through 4, but since the eye coordinates were locked in at the last minute it gave the system the optimum chance at locking in eye coordinates that were the most accurate.

In some embodiments, processing steps in the calculation of the bird's eye position as close as possible to the nozzle are as follows. As the bird's eye (target area) moves down the belt the X, Y, and Z coordinates are determined. The frame rate of the hardware determines the next time new coordinates can be acquired. By comparing any two successive X, Y, and Z coordinates, their relative positions with respect to one another can be determined. For a bird that is stationary and not moving the difference in coordinates is defined by the distance traversed by the bird down the belt. This expected location (for a non-moving bird) can be compared to the actual location of the bird between Positions. For example the difference in X, Y, and Z coordinates between Positions 3 and 4. In this example, this difference would indicate that in addition to translating down the belt due to being on a conveyor the bird is also moving downwards. The latest X, Y and Z coordinates that can be locked in are the coordinates from position 4, but by determining the bird is moving downwards between positions 3 and 4, this same amount of movement can predictively be applied to the targeting position at Position 5. This type of algorithm would predictively accommodate the movement of a bird during the time from imaging to spray by utilizing the direction of movement the bird was in moments before and continuing on in that movement. This can be further refined to string together multiple position points to create predictive accelerations or decelerations. This predictive positioning can be accomplished independently for each eye in all three axes without departing from the scope of the present inventive concept.

It will be understood that embodiments illustrated in FIG. 14 assumes shutter speed in the algorithm is equal to frame rate. If the algorithm is faster than frame rate, the opportunity may exist for a slightly more up to date final X, Y and Z position.

In some embodiments, rather than directly measuring the position of the eyes of the bird, the eyes of the bird may be tracked in space as they move down the belt and wait to lock the eye positions until as near as possible to the spray station. This may be accomplished, for example, with a simple thresholding or blob detection algorithm and an array of 2D cameras.

As is clear from the discussion above, some aspects of the present inventive concept may be implemented by a data processing system and a location module including a scanning system, buffer, scripts and the like. The data processing system may be included at any module of the system without departing from the scope of the preset inventive concept. Exemplary embodiments of a data processing system 1530 configured in accordance with embodiments of the present inventive concept will be discussed with respect to FIG. 15. The data processing system 1530 may include a user interface 1544, including, for example, input device(s) such as a keyboard or keypad, a display, a speaker and/or microphone, and a memory 1536 that communicate with a processor 1538. The data processing system 1530 may further include I/O data port(s) 1546 that also communicates with the processor 1538. The I/O data ports 1546 can be used to transfer information between the data processing system 1530 and another computer system or a network using, for example, an Internet Protocol (IP) connection. These components may be conventional components such as those used in many conventional data processing systems, which may be configured to operate as described herein.

As illustrated, the processor 1538 communicates with a location module 1560 and a scanning system 1565 that perform various aspects of the present inventive concept discussed above. For example, the scanning system 1565 is used to obtain the scans discussed above with respect to the various embodiments and some of these scans may be stored as “slices: in the buffer 1570. As further illustrated, the location module 1560 has access to the scanning system 1565 and the buffer 1570 and may use these scans to determine target location(s) and to calculate a spray timing as discussed above. Custom scripts 1575 may be used to analyze the scans and adjust a nozzle and spray according thereto.

Some example tests were performed using systems and methods according to embodiments discussed herein. Results of some of these tests will be discussed herein. It will be understood that the parameters used in these tests and the results thereof are provided for example only and, therefore, embodiments of the present inventive concept are not limited thereto.

In some embodiments, systems and methods in accordance with embodiments discussed herein may produce an eye/face targeting percentage of at least 85% of birds sprayed in the eye or face. A particular test run included 22,000 birds tested across two hatcheries and produced an eye/face targeting percentage of at least about 92.7% eye/face targeting. In this example, the speed of the belt was at least 15 inches/second, for example, 45 inches/second and a spray delivery volume of no greater than 220 ul. In some embodiments, the delivery volume may be no greater than 120 ul/chick. Spraying the chicks at this spray delivery volume may provide a benefit in terms of minimizing chick chilling which can adversely affect chick health.

In some embodiments, a multi-stream nozzle spray may be used in place of a cone angle spray to effectively control pattern size and vaccine pattern area across the width of the belt. In some embodiments, a multi-nozzle bank may be selected from to fire parallel streams to the target region, for example, one or both of the bird's eyes. This may provide a maximum positional accommodation and vaccine efficacy independent of bird distance from the spray nozzle.

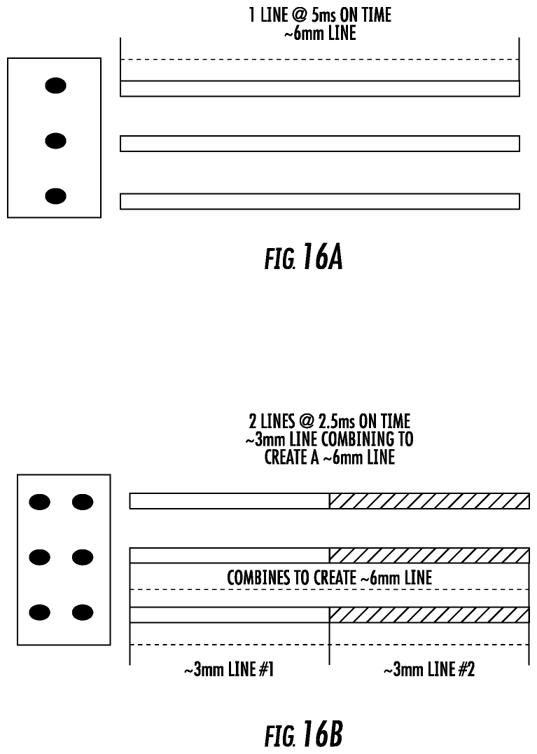

Embodiments including multi-nozzle spray in various patterns are illustrated, for example, in FIGS. 16A and 16B. As shown, in some embodiments, the stream may be applied by multiple nozzles oriented along the length of the belt such that as they spray the orientation of the nozzles in space helps to create the pattern on the bird. This has the benefit of being able to dispense a dose of a given pattern size without having to wait for the target to move along the conveyor to create the pattern decreasing the amount of time from imaging to finish of spray, thus decreasing the opportunity for chick movement.

In particular, as illustrated in FIGS. 16A and 16B, delivering a 6 mm pattern on an eye of the subject bird at a belt speed of, for example, 30 inches/second equates to 5 ms of on-time for the valve. At very fast image acquisition, and algorithm speeds the time to dose the vaccine becomes a substantial portion of the overall wait time from imaging to vaccine application finish. FIGS. 16A and 16B illustrate how firing different portions of the stream at the same time to make up the full pattern on the bird saves time by reducing the amount of time it takes to dose a full pattern on the bird. It will be understood that although the figures show the stream broken up into two sections, embodiments are not limited thereto. The stream can be broken up into a full dot matrix where once actuated the full shape of the pattern is flying through the air at once hitting the bird nearly simultaneously. Using concepts illustrated in FIGS. 16A and 16B, would allow creating of any pattern size or shape, but at the same time eliminating nearly all the time associated with dosing. In other words, instead of turning a valve on and waiting for the belt to move the bird through the spray, the pattern would fly through the air at the bird and upon impact, the pattern would create the desired shape.

In some embodiments, the parameters (FIG. 2) may include a Bird Time to Move (T) of ≤200 ms (amount of time the bird has to move between imaging and spray). In some embodiments, the Bird Time to Move (T) may be about 87 ms average, within a range of from about 74 ms to about 118 ms. Referring to FIG. 11, in some embodiments, the processing steps from block 1105 to the end may have a software response time of <50 ms (Image Analysis to calculation completion). In some embodiments, the average system response time may be about 25 ms, with a range of about 20 ms to about 35 ms. In some embodiments, the average system response time may be about 60 ms, with a range of about 40 ms to about 85 ms. In some embodiments, the average system response time may be about 35 ms, with a range of about 23 ms to about 45 ms. In some embodiments, the average system response time may be about 24 ms, with a range of about 14 ms to about 32 ms. In some embodiments, the average system response time may be about 17 ms, with a range of about 10 ms to about 25 ms. In some embodiments, the average system response time may be about 9 ms, with a range of about 5 ms to about 12 ms.

As discussed briefly above, some embodiments of the present inventive concept provide methods, systems and computer program products for adjusting for positional changes in the subject to provide accurate delivery of a substance (spray) to a target zone of the subject (chick eyes). Furthermore, some embodiments provide strategies for improving the effectiveness of the delivered dose and decreasing a time from scanning to delivery. Thus, embodiments of the present inventive concept provide improved accuracy as well as decreased timing of the spray.

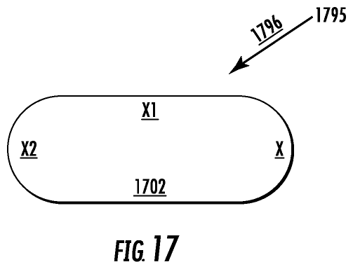

As discussed above, some embodiments of the present inventive concept may be used to deliver a substance via spray to, for example, a bird. However, as discussed, embodiments of the present inventive concept are not limited to this configuration. Referring now to FIG. 17, a generic subject that receives a substance in accordance with various embodiments of the present inventive concept will be discussed. In particular, example embodiments of the present inventive concept are provided herein as having a bird as the subject, however, embodiments of the present inventive concept are not limited thereto. As illustrated in FIG. 17, a subject 1702 is shown having various target regions X, X1 and X2. Although only one subject 1702 is shown in FIG. 17 having only three target regions, embodiments of the present inventive concept are not limited thereto. More than one subject having more or less than three target regions may be present.

The subject 1702 may be, for example, any type of poultry including, but not limited to, chicken, turkey, duck, geese, quail, pheasant, guineas, guinea fowl, peafowl, partridge, pigeon, emu, ostrich, exotic birds, and the like. The subject may also be a non-poultry livestock, such as cows, ox, sheep, donkey, goat, llama, horses, and pigs (swine) as well as aquatic animals. The target regions X, X1 and X2 may be any region on the subject 1702 that is fit for receiving the substance. For example, the target region may be the mouth or snout, neck, rump, eyes or nasal portions of the subject 1702 or even an underbelly of an aquatic animal without departing from the scope of the present inventive concept.

Algorithms and methods similar to those discussed above with respect to FIGS. 1 through 16 may be used to determine the position and/or orientation of the subject and its associated target region(s). Once the position and/or orientation of the subject 1702 is determined, the substance 1795 may be delivered using one of various methods 1796. The substance being delivered may be, for example: vaccinations against Newcastle disease, infectious bronchitis virus, E coli, salmonella, coccidia, camplyobactor, Marek's disease, Infectious bursal disease, Tenosynovitis, Encephalomyelitis, Fowlpox, Chicken infectious anemia, Laryngotracheitis, Fowl cholera, Mycoplasma gallispticum, ND B1-B1, LaSota, DW, Hemorrhagic enteritis, SC, Erysipelas, Riemerella anatipestifer, Duck viral hepatitis, and Duck viral enteritis. However, embodiments are not limited thereto. Although embodiments of the present inventive concept discussed above focus on spray delivery methods, the substance may be delivered using, for example, a needle or needleless injection or any other possible delivery system without departing from the present inventive concept.

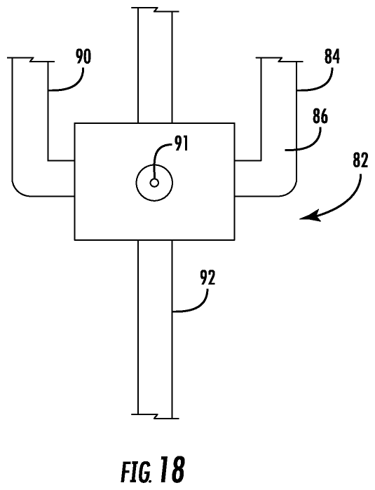

For example, an automated injection system illustrated in FIG. 18 may be used to deliver the substance after the subject has been scanned. As illustrated in FIG. 18, the automated injection system 82 includes a reservoir 84 filled with a substance 86, such as a vaccine, drug, biologic or other medicament used to treat the subject. The injection system 82 also includes a pressurized gas supply 90 and an injection head 91. Pressurized gas may be delivered to the automatic injection system 82 via pre pressurized gas capsules or alternatively via a gas plumbing attached to a centralized compressor.

The injection system 82 may be adjustably mounted to a frame 92 that allows for automatic adjustment to the height, depth and length of the injection system. The frame 92 is fixedly mounted to a fixed structure. The automatic adjustability of the injection system 82 is achieved by mechanisms that can automatically and remotely adjust the height, width and depth of the injection system 82 relative to the position of the subject and the target regions X, X1 and X2 thereon. The pressurized gas supply 90 may be used to deliver the substance 86 within the reservoir 84 into the subject. It is appreciated that the control of the pressurized gas supply 90 and substance 86 are understood by those skilled in the art of needle-free delivery devices. Thus, the injection may be a needle or needleless. It will be understood that the injection system illustrated in FIG. 18 is provided as an example only and, therefore, embodiments of the present inventive concept are not limited thereto.

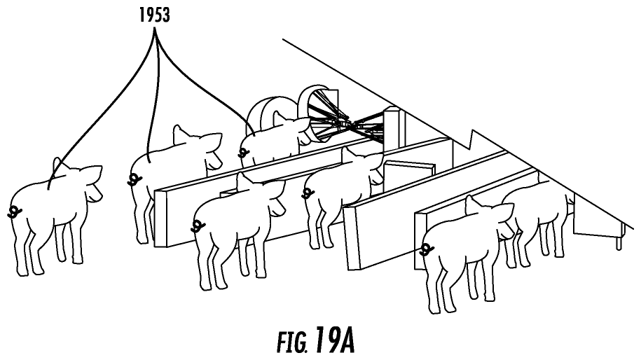

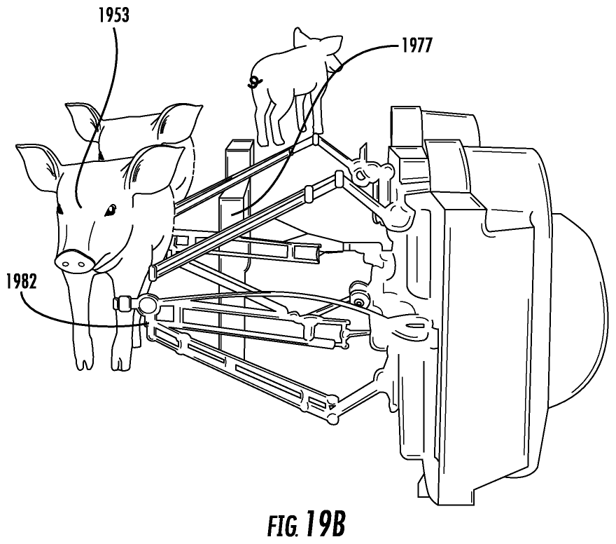

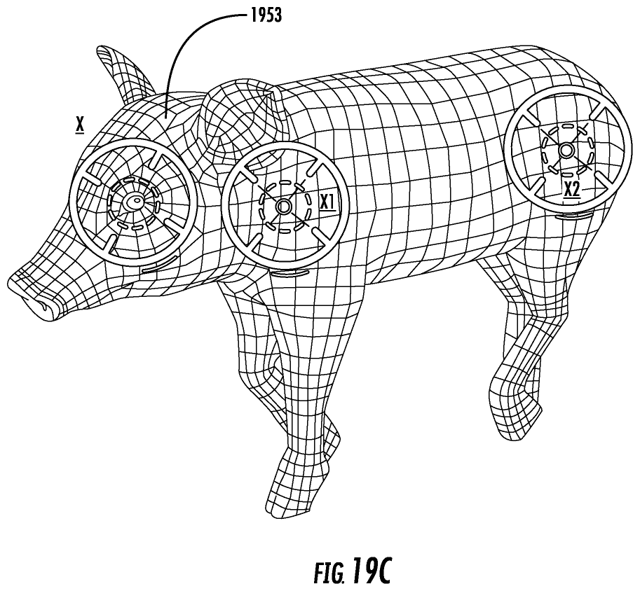

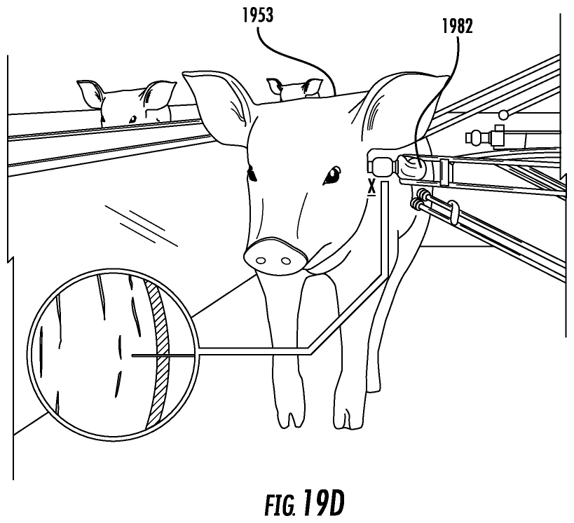

In particular, methods for delivering a substance to a subject in accordance with embodiments discussed herein may be used to deliver a substance to swine as illustrated in FIGS. 19A through 19D. As illustrated in FIG. 19A, the subjects in these illustrated embodiments are swine 1953. As further illustrated, the swine 1953 are lined up in a series of long lines separated by wall, similar to embodiments discussed above with respect to FIGS. 1B and 1C for the chicks. As illustrated in FIG. 19B, as the swine 1953 approach the injection system (or spray system) 1982 the swine are scanned 1977 in accordance with embodiments discussed herein to locate the target zones X, X1 and X2 (FIG. 19C) on the swine 1953. It will be understood that the swine 1953 may not move the same way the chicks move as discussed above. Accordingly, the algorithm may be adjusted for locating target zones X, X1 and X2 on the swine 1953 without departing from the scope of the present inventive concept. As illustrated in FIG. 19D, once one or more target zones X, X1 and X2 are located, the injection system 1982 may be used to inject the substance into the swine 1953.

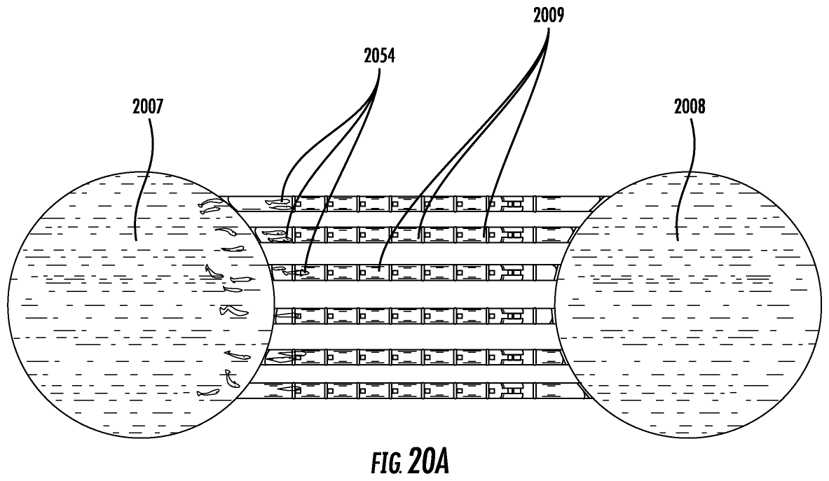

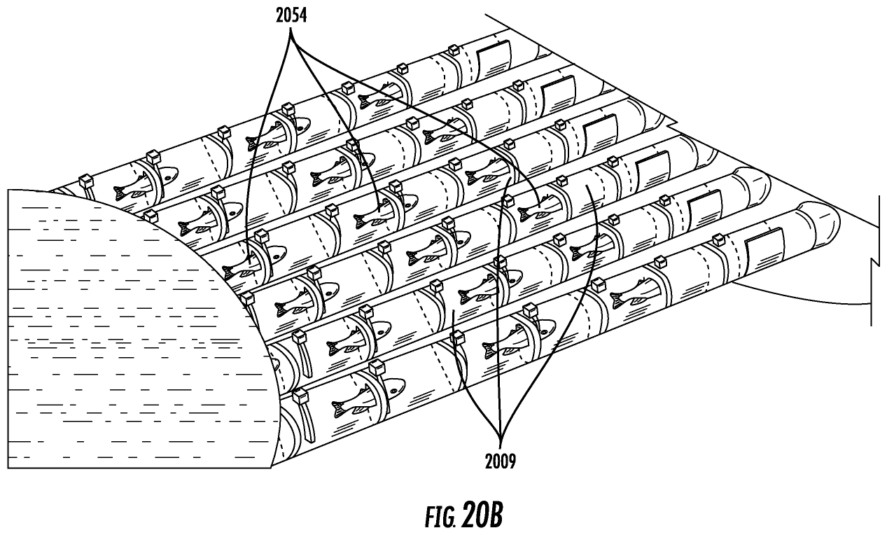

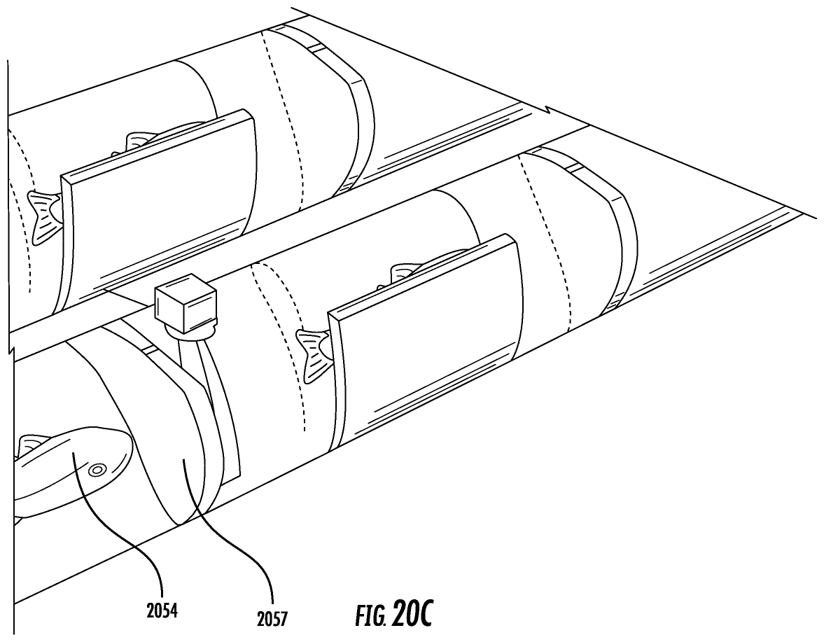

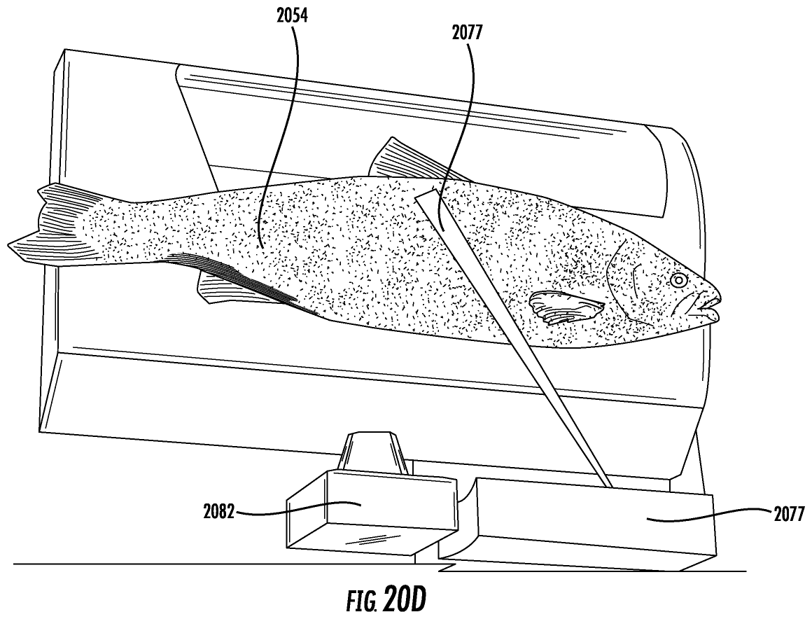

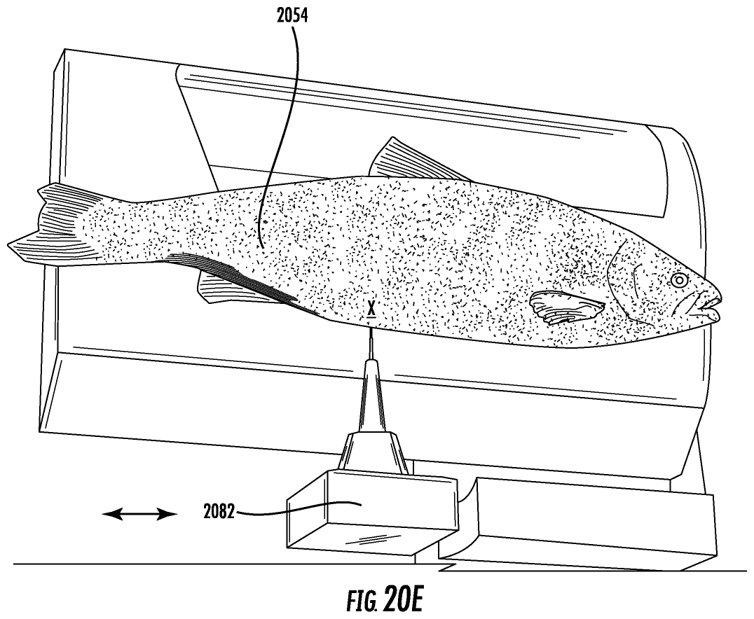

Similarly, in some embodiments, methods for delivering a substance to a subject in accordance with embodiments discussed herein may be used to deliver a substance to a fish as illustrated in FIGS. 20A through 20E. As illustrated in FIG. 20A, the fish 2054 swim from a first pool 2007 to a second pool 2008 through a series of tubes 2009. An exploded view of the fish 2054 swimming in the tubes 2009 is provided in FIG. 20B. As illustrated in FIG. 20c, as the fish 2054 swim through the tubes from the first pool 2007 to the second pool 2008, they are captured using, for example, metal plates 2057. It will be understood that embodiments of the present inventive concept are not limited to this configuration, other methods of isolating each fish may include inflatable bladders located fore and aft of the fish. Once captured, as shown in FIG. 20D, the fish 2054 is scanned 2077 in accordance with embodiments discussed herein to locate the target zones X (FIG. 20E) on the fish 2054. It will be understood that the fish 2054 may not move the same way the chicks move as discussed above. Accordingly, the algorithm may be adjusted for locating target zones X, X1 and X2 on the fish 2054 without departing from the scope of the present inventive concept. As illustrated in FIG. 20E, once one or more target zones X, X1 and X2 are located on the fish, the injection system 2082 may be used to inject the substance into the fish 2054. As illustrated, the scanning system 2077 and the injection system 2082 may move from side to side such that the injection can be delivered to the target X. The fish 2054 are then released into the second pool 2008.

Although specific embodiments of chicks, swine and fish are discussed herein, embodiments of the present inventive concept are not limited to these examples. Any subject discussed above may be delivered a substance as discussed herein without departing from the scope of the present inventive concept.

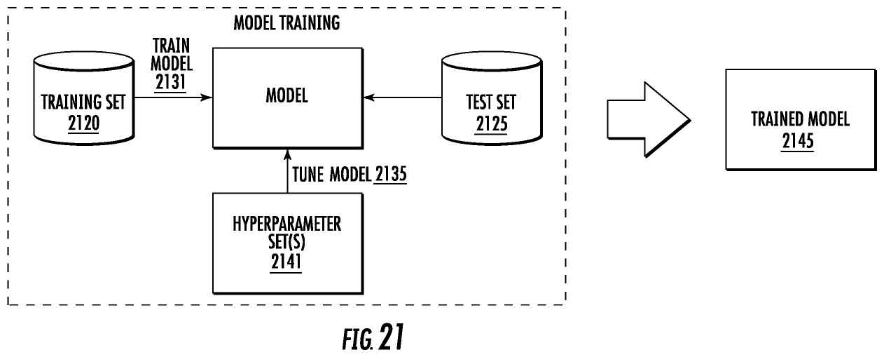

As discussed above, some embodiments of the present inventive concept utilize machine learning and/or artificial intelligence. Referring now to FIG. 21, a diagram illustrating an example of training a machine learning model in connection with present disclosure will be discussed. The machine learning model training described herein may be performed using a machine learning system. The machine learning system may include or may be included in a computing device, a server, a cloud computing environment, or the like.

A machine learning model may be trained using a set of observations. The set of observations may be obtained and/or input from historical data, such as data gathered during one or more processes described herein. For example, the set of observations may include data gathered about a position of a bird on a belt relative to the spray nozzle, as described elsewhere herein. In some implementations, the machine learning system may receive the set of observations (e.g., as input) from the location module 160 (FIG. 1A) or from a storage device.

A feature set may be derived from the set of observations. The feature set may include a set of variables. A variable may be referred to as a feature. A specific observation may include a set of variable values corresponding to the set of variables. A set of variable values may be specific to an observation. In some cases, different observations may be associated with different sets of variable values, sometimes referred to as feature values.

In some implementations, the machine learning system may determine variables for a set of observations and/or variable values for a specific observation based on input received from location module 160. For example, the machine learning system may identify a feature set (e.g., one or more features and/or corresponding feature values) from structured data input to the machine learning system, such as by extracting data from a particular column of a table, extracting data from a particular field of a form and/or a message, and/or extracting data received in a structured data format. Additionally, or alternatively, the machine learning system may receive input from an operator to determine features and/or feature values.

In some implementations, the machine learning system may perform natural language processing and/or another feature identification technique to extract features (e.g., variables) and/or feature values (e.g., variable values) from text (e.g., unstructured data) input to the machine learning system, such as by identifying keywords and/or values associated with those keywords from the text.

As an example, a feature set for a set of observations may include a first position of the bird on the belt, a second position of the bird on the belt, and so on. These features and feature values are provided as examples and may differ in other examples. For example, the feature set may include one or more of the following features: position of birds' eyes, height of birds eyes, relative position of the bird on the belt, etc. In some implementations, the machine learning system may pre-process and/or perform dimensionality reduction to reduce the feature set and/or combine features of the feature set to a minimum feature set. A machine learning model may be trained on the minimum feature set, thereby conserving resources of the machine learning system (e.g., processing resources and/or memory resources) used to train the machine learning model.

The set of observations may be associated with a target variable. The target variable may represent a variable having a numeric value (e.g., an integer value or a floating point value), may represent a variable having a numeric value that falls within a range of values or has some discrete possible values, may represent a variable that is selectable from one of multiple options (e.g., one of multiples classes, classifications, or labels), or may represent a variable having a Boolean value (e.g., 0 or 1, True or False, Yes or No), among other examples. A target variable may be associated with a target variable value, and a target variable value may be specific to an observation. In some cases, different observations may be associated with different target variable values. The target variable may be the position of the bird, which has an XYZ value (3D coordinate value) for the first observation. The feature set and target variable described above are provided as examples, and other examples may differ from what is described above.

The target variable may represent a value that a machine learning model is being trained to predict, and the feature set may represent the variables that are input to a trained machine learning model to predict a value for the target variable. The set of observations may include target variable values so that the machine learning model can be trained to recognize patterns in the feature set that lead to a target variable value. A machine learning model that is trained to predict a target variable value may be referred to as a supervised learning model or a predictive model. When the target variable is associated with continuous target variable values (e.g., a range of numbers), the machine learning model may employ a regression technique. When the target variable is associated with categorical target variable values (e.g., classes or labels), the machine learning model may employ a classification technique.

In some implementations, the machine learning model may be trained on a set of observations that do not include a target variable (or that include a target variable, but the machine learning model is not being executed to predict the target variable). This may be referred to as an unsupervised learning model, an automated data analysis model, or an automated signal extraction model. In this case, the machine learning model may learn patterns from the set of observations without labeling or supervision, and may provide output that indicates such patterns, such as by using clustering and/or association to identify related groups of items within the set of observations.

As shown in FIG. 21, the machine learning system may partition the set of observations into a training set 2120 that includes a first subset of observations of the set of observations, and a test set 2125 that includes a second subset of observations of the set of observations. The training set 2120 may be used to train (e.g., fit or tune) the machine learning model, while the test set 2125 may be used to evaluate a machine learning model that is trained using the training set 2120. For example, for supervised learning, the test set 2125 may be used for initial model training using the first subset of observations, and the test set 2125 may be used to test whether the trained model accurately predicts target variables in the second subset of observations. In some implementations, the machine learning system may partition the set of observations into the training set 2120 and the test set 2125 by including a first portion or a first percentage of the set of observations in the training set 2120 (e.g., 75%, 80%, or 85%, among other examples) and including a second portion or a second percentage of the set of observations in the test set 2125 (e.g., 25%, 20%, or 15%, among other examples). In some implementations, the machine learning system may randomly select observations to be included in the training set 2120 and/or the test set 2125.

As shown by reference number 2131, the machine learning system may train a machine learning model using the training set 2120. This training may include executing, by the machine learning system, a machine learning algorithm to determine a set of model parameters based on the training set 2120. In some implementations, the machine learning algorithm may include a regression algorithm (e.g., linear regression or logistic regression), which may include a regularized regression algorithm (e.g., Lasso regression, Ridge regression, or Elastic-Net regression). Additionally, or alternatively, the machine learning algorithm may include a decision tree algorithm, which may include a tree ensemble algorithm (e.g., generated using bagging and/or boosting), a random forest algorithm, or a boosted trees algorithm. A model parameter may include an attribute of a machine learning model that is learned from data input into the model (e.g., the training set 2120). For example, for a regression algorithm, a model parameter may include a regression coefficient (e.g., a weight). For a decision tree algorithm, a model parameter may include a decision tree split location, as an example.

As shown by reference number 2135, the machine learning system may use one or more hyperparameter sets 2141 to tune the machine learning model. A hyperparameter may include a structural parameter that controls execution of a machine learning algorithm by the machine learning system, such as a constraint applied to the machine learning algorithm. Unlike a model parameter, a hyperparameter is not learned from data input into the model. An example hyperparameter for a regularized regression algorithm includes a strength (e.g., a weight) of a penalty applied to a regression coefficient to mitigate overfitting of the machine learning model to the training set 2120. The penalty may be applied based on a size of a coefficient value (e.g., for Lasso regression, such as to penalize large coefficient values), may be applied based on a squared size of a coefficient value (e.g., for Ridge regression, such as to penalize large squared coefficient values), may be applied based on a ratio of the size and the squared size (e.g., for Elastic-Net regression), and/or may be applied by setting one or more feature values to zero (e.g., for automatic feature selection). Example hyperparameters for a decision tree algorithm include a tree ensemble technique to be applied (e.g., bagging, boosting, a random forest algorithm, and/or a boosted trees algorithm), a number of features to evaluate, a number of observations to use, a maximum depth of each decision tree (e.g., a number of branches permitted for the decision tree), or a number of decision trees to include in a random forest algorithm.

To train a machine learning model, the machine learning system may identify a set of machine learning algorithms to be trained (e.g., based on operator input that identifies the one or more machine learning algorithms and/or based on random selection of a set of machine learning algorithms), and may train the set of machine learning algorithms (e.g., independently for each machine learning algorithm in the set) using the training set 2120. The machine learning system may tune each machine learning algorithm using one or more hyperparameter sets 2141 (e.g., based on operator input that identifies hyperparameter sets 2141 to be used and/or based on randomly generating hyperparameter values). The machine learning system may train a particular machine learning model using a specific machine learning algorithm and a corresponding hyperparameter set 2141. In some implementations, the machine learning system may train multiple machine learning models to generate a set of model parameters for each machine learning model, where each machine learning model corresponds to a different combination of a machine learning algorithm and a hyperparameter set 2141 for that machine learning algorithm.

In some implementations, the machine learning system may perform cross-validation when training a machine learning model. Cross validation can be used to obtain a reliable estimate of machine learning model performance using only the training set 2120, and without using the test set 2125, such as by splitting the training set 2120 into a number of groups (e.g., based on operator input that identifies the number of groups and/or based on randomly selecting a number of groups) and using those groups to estimate model performance. For example, using k-fold cross-validation, observations in the training set 2120 may be split into k groups (e.g., in order or at random). For a training procedure, one group may be marked as a hold-out group, and the remaining groups may be marked as training groups. For the training procedure, the machine learning system may train a machine learning model on the training groups and then test the machine learning model on the hold-out group to generate a cross-validation score. The machine learning system may repeat this training procedure using different hold-out groups and different test groups to generate a cross-validation score for each training procedure. In some implementations, the machine learning system may independently train the machine learning model k times, with each individual group being used as a hold-out group once and being used as a training group k−1 times. The machine learning system may combine the cross-validation scores for each training procedure to generate an overall cross-validation score for the machine learning model. The overall cross-validation score may include, for example, an average cross-validation score (e.g., across all training procedures), a standard deviation across cross-validation scores, or a standard error across cross-validation scores.

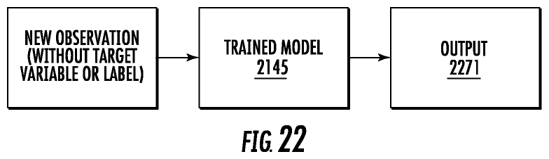

In some implementations, the machine learning system may perform cross-validation when training a machine learning model by splitting the training set into a number of groups (e.g., based on operator input that identifies the number of groups and/or based on randomly selecting a number of groups). The machine learning system may perform multiple training procedures and may generate a cross-validation score for each training procedure. The machine learning system may generate an overall cross-validation score for each hyperparameter set 2141 associated with a particular machine learning algorithm. The machine learning system may compare the overall cross-validation scores for different hyperparameter sets 2141 associated with the particular machine learning algorithm, and may select the hyperparameter set 2141 with the best (e.g., highest accuracy, lowest error, or closest to a desired threshold) overall cross-validation score for training the machine learning model. The machine learning system may then train the machine learning model using the selected hyperparameter set 2141, without cross-validation (e.g., using all of data in the training set 2120 without any hold-out groups), to generate a single machine learning model for a particular machine learning algorithm. The machine learning system may then test this machine learning model using the test set 2125 to generate a performance score, such as a mean squared error (e.g., for regression), a mean absolute error (e.g., for regression), or an area under receiver operating characteristic curve (e.g., for classification). If the machine learning model performs adequately (e.g., with a performance score that satisfies a threshold), then the machine learning system may store that machine learning model as a trained machine learning model 2145 to be used to analyze new observations, as described below in connection with FIG. 22.

In some implementations, the machine learning system may perform cross-validation, as described above, for multiple machine learning algorithms (e.g., independently), such as a regularized regression algorithm, different types of regularized regression algorithms, a decision tree algorithm, or different types of decision tree algorithms. Based on performing cross-validation for multiple machine learning algorithms, the machine learning system may generate multiple machine learning models, where each machine learning model has the best overall cross-validation score for a corresponding machine learning algorithm. The machine learning system may then train each machine learning model using the entire training set 2120 (e.g., without cross-validation), and may test each machine learning model using the test set to generate a corresponding performance score for each machine learning model. The machine learning model may compare the performance scores for each machine learning model, and may select the machine learning model with the best (e.g., highest accuracy, lowest error, or closest to a desired threshold) performance score as the trained machine learning model 2145.

As indicated above, FIG. 21 is provided as an example. Other examples may differ from what is described in connection with FIG. 21. For example, the machine learning model may be trained using a different process than what is described in connection with FIG. 21. Additionally, or alternatively, the machine learning model may employ a different machine learning algorithm than what is described in connection with FIG. 21, such as a Bayesian estimation algorithm, a k-nearest neighbor algorithm, an a priori algorithm, a k-means algorithm, a support vector machine algorithm, a neural network algorithm (e.g., a convolutional neural network algorithm), and/or a deep learning algorithm.

FIG. 22 is a diagram illustrating an example of applying a trained machine learning model to a new observation associated with delivering a substance to a subject. The new observation may be input to a machine learning system that stores a trained machine learning model 2145, such as the trained machine learning model 2145 described above in connection with FIG. 21. The machine learning system may include or may be included in a computing device, a server, or a cloud computing environment.

The machine learning system may receive a new observation (or a set of new observations), and may input the new observation to the machine learning model. As shown, the new observation may include a first feature, a second feature, a third feature and the like. The machine learning system may apply the trained machine learning model 2145 to the new observation to generate an output 2271 (e.g., a result). The type of output may depend on the type of machine learning model and/or the type of machine learning task being performed. For example, the output 2271 may include a predicted (e.g., estimated) value of target variable (e.g., a value within a continuous range of values, a discrete value, a label, a class, or a classification), such as when supervised learning is employed. Additionally, or alternatively, the output 2271 may include information that identifies a cluster to which the new observation belongs and/or information that indicates a degree of similarity between the new observation and one or more prior observations (e.g., which may have previously been new observations input to the machine learning model and/or observations used to train the machine learning model), such as when unsupervised learning is employed.

In some implementations, the trained machine learning model 2145 may predict an XYZ value of a location of the bird. Based on this prediction (e.g., based on the value having a particular label or classification or based on the value satisfying or failing to satisfy a threshold), the machine learning system may provide a recommendation and/or output for determination of a recommendation, such as providing an indication that the substance should be delivered to the bird. Additionally, or alternatively, the machine learning system may perform an automated action and/or may cause an automated action to be performed (e.g., by instructing another device to perform the automated action). In some implementations, the recommendation and/or the automated action may be based on the target variable value having a particular label (e.g., classification or categorization) and/or may be based on whether the target variable value satisfies one or more threshold (e.g., whether the target variable value is greater than a threshold, is less than a threshold, is equal to a threshold, or falls within a range of threshold values).

In this way, the machine learning system may apply a rigorous and automated process to determine a location of a bird and when to deliver a substance thereto. The machine learning system enables recognition and/or identification of tens, hundreds, thousands, or millions of features and/or feature values for tens, hundreds, thousands, or millions of observations, thereby increasing accuracy and consistency and reducing delay associated with chick vaccination relative to the required resources (e.g., computing or manual) to be allocated for tens, hundreds, or thousands of operators to manually vaccinate birds.

As indicated above, FIG. 22 is provided as an example. Other examples may differ from what is described in connection with FIG. 22.

The aforementioned flow logic and/or methods show the functionality and operation of various services and applications described herein. If embodied in software, each block may represent a module, segment, or portion of code that includes program instructions to implement the specified logical function(s). The program instructions may be embodied in the form of source code that includes human-readable statements written in a programming language or machine code that includes numerical instructions recognizable by a suitable execution system such as a processor in a computer system or other system. The machine code may be converted from the source code, etc. Other suitable types of code include compiled code, interpreted code, executable code, static code, dynamic code, object-oriented code, visual code, and the like. The examples are not limited in this context.

If embodied in hardware, each block may represent a circuit or a number of interconnected circuits to implement the specified logical function(s). A circuit can include any of various commercially available processors, including without limitation an AMD® Athlon®, Duron® and Opteron® processors; ARM® application, embedded and secure processors; IBM® and Motorola® DragonBall® and PowerPC® processors; IBM and Sony® Cell processors; Qualcomm® Snapdragon®; Intel® Celeron®, Core (2) Duo®, Core i3, Core i5, Core i7, Itanium®, Pentium®, Xeon®, Atom® and XScale® processors; Nvidia Jetson®-class processors (e.g. Xavier and Orin families) and similar processors. Other types of multi-core processors and other multi-processor architectures may also be employed as part of the circuitry. According to some examples, circuitry may also include an application specific integrated circuit (ASIC) or a field programmable gate array (FPGA), and modules may be implemented as hardware elements of the ASIC or the FPGA. Further, embodiments may be provided in the form of a chip, chipset or package.

Although the aforementioned flow logic and/or methods each show a specific order of execution, it is understood that the order of execution may differ from that which is depicted. Also, operations shown in succession in the flowcharts may be able to be executed concurrently or with partial concurrence. Further, in some embodiments, one or more of the operations may be skipped or omitted. In addition, any number of counters, state variables, warning semaphores, or messages might be added to the logical flows or methods described herein, for purposes of enhanced utility, accounting, performance measurement, or providing troubleshooting aids, etc. It is understood that all such variations are within the scope of the present disclosure. Moreover, not all operations illustrated in a flow logic or method may be required for a novel implementation.

Where any operation or component discussed herein is implemented in the form of software, any one of a number of programming languages may be employed such as, for example, C, C++, C#, Objective C, Java, Javascript, Perl, PHP, Visual Basic, Python, Ruby, Delphi, Flash, or other programming languages. Software components are stored in a memory and are executable by a processor. In this respect, the term “executable” means a program file that is in a form that can ultimately be run by a processor. Examples of executable programs may be, for example, a compiled program that can be translated into machine code in a format that can be loaded into a random access portion of a memory and run by a processor, source code that may be expressed in proper format such as object code that is capable of being loaded into a random access portion of a memory and executed by a processor, or source code that may be interpreted by another executable program to generate instructions in a random access portion of a memory to be executed by a processor, etc. An executable program may be stored in any portion or component of a memory. In the context of the present disclosure, a “computer-readable medium” can be any medium (e.g., memory) that can contain, store, or maintain the logic or application described herein for use by or in connection with the instruction execution system.

A memory is defined herein as an article of manufacture and including volatile and/or non-volatile memory, removable and/or non-removable memory, erasable and/or non-erasable memory, writeable and/or re-writeable memory, and so forth. Volatile components are those that do not retain data values upon loss of power. Nonvolatile components are those that retain data upon a loss of power. Thus, a memory may include, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may include, for example, static random access memory (SRAM), dynamic random access memory (DRAM), or magnetic random access memory (MRAM) and other such devices. The ROM may include, for example, a programmable read-only memory (PROM), an erasable programmable read-only memory (EPROM), an electrically erasable programmable read-only memory (EEPROM), or other like memory device.

The devices described herein may include multiple processors and multiple memories that operate in parallel processing circuits, respectively. In such a case, a local interface, such as a communication bus, may facilitate communication between any two of the multiple processors, between any processor and any of the memories, or between any two of the memories, etc. A local interface may include additional systems designed to coordinate this communication, including, for example, performing load balancing. A processor may be of electrical or of some other available construction.

It should be emphasized that the above-described embodiments of the present disclosure are merely possible examples of implementations set forth for a clear understanding of the principles of the disclosure. It is, of course, not possible to describe every conceivable combination of components and/or methodologies, but one of ordinary skill in the art may recognize that many further combinations and permutations are possible. That is, many variations and modifications may be made to the above-described embodiment(s) without departing substantially from the spirit and principles of the disclosure. All such modifications and variations are intended to be included herein within the scope of this disclosure and protected by the following claims.

That which is claimed is:

1. A method for accurately administering a substance to a subject in motion, the method comprising:
obtaining one or more scans of the subject, the subject having at least one defined target region thereon for delivery of the substance;
calculating a three dimensional position of the subject in motion based on the obtained one or more scans of the subject, the three dimensional position including X, Y, and Z coordinates defining the three dimensional position;
calculating a timing adjustment based on the calculated three dimensional position of the subject in motion; and
adjusting a timing of the delivery of the substance to the at least one defined target region on the subject using the calculated timing adjustment,
wherein the obtaining, calculating the three dimensional position, calculating the timing adjustment and the adjusting the timing of the delivery are performed by at least one processor.

2. The method of claim 1, wherein at least 85% of the subjects receive delivery of the substance in the at least one defined target region.

3. The method of claim 2, wherein greater than 92% of the subjects receive delivery of the substance in the at least one defined target region.

4. The method of claim 1, wherein obtaining one or more scans comprises obtaining a single scan of a whole subject in motion.

5. The method of claim 1, wherein obtaining comprises:
obtaining a first slice scan of the subject in motion, the first slice scan being a scan of less than a whole subject;
determining if the first slice scan exceeds a threshold indicating that an entire defined target area is visible in the first slice scan;
if it is determined that the entire defined target area is visible in the first slice scan, proceeding to calculating the three dimensional position of the subject in motion based on the first slice scan;
if it is determined that the first slice scan does not exceed the threshold, obtaining an additional slice scan;
combining the first slice scan and the additional slice scan to provide a combined scan;
determining if the combined scan exceeds the threshold;
repeating the obtaining and combining steps until it is determined that the threshold has been exceeded; and
proceeding to calculating the three dimensional position of the subject in motion based on the combined scan when it is determined that the threshold indicating that the entire defined target area is visible is exceeded.

6. The method of claim 1, further comprising:
calculating a nozzle adjustment factor based on the calculated three dimensional position of the subject in motion; and
adjusting a position of at least one nozzle used to administer the substance based on the calculated nozzle adjustment factor.

7. The method of claim 6, wherein calculating the timing adjustment and the nozzle adjustment factor comprises calculating the timing adjustment and the nozzle adjustment factor based on one or more of the following: a velocity of a conveyor belt on which the subject is traveling (v_{b}); a time of flight (TofF) before the substance is delivered to the subject; a speed at which the substance is delivered (vₛ); a distance the at least one defined target region is from a nozzle delivering the substance (dₜₙ); and a width of the conveyor belt (w_{c}).

8. The method of claim 6, further comprising administering the substance to the at least one defined target region of the subject at a time and position altered by the nozzle adjustment factor and/or the timing adjustment.

9. The method of claim 6, wherein the at least one nozzle comprise one or more nozzle banks.

10. The method of claim 1, wherein the subject is a bird and the at least one defined target region is one or more of a mucosa in one or more eyes of the bird, an area around one or more eyes of the bird, nostrils of the bird, mouth of the bird, and/or any orifice on a head of the bird that leads to the gut and/or respiratory tract.

11. The method of claim 1, wherein the subject is a swine and wherein the method further comprises delivering the substance to the swine using at least one needle or needle free injector.

12. The method of claim 1, wherein the substance is delivered in a volume no greater than 120 ul/subject.

13. The method of claim 1, wherein the method further comprising delivering the substance to the subject from a day of hatch to chicks having an age of five days.

14. The method of claim 1, wherein the subject is any human or animal that receives the substance.

15. A system for accurately administering a substance to a subject in motion, the system comprising:
a scanning system that obtains one or more scans of the subject, the subject having at least one defined target region thereon for delivery of the substance; and
a location module that:
calculates a three dimensional position of the subject in motion based on the obtained one or more scans of the subject, the three dimensional position including X, Y, and Z coordinates defining the three dimensional position;
calculates a timing adjustment based on the calculated three dimensional position of the subject in motion; and
adjusts a timing of the delivery of the substance to the at least one defined target region on the subject using the calculated timing adjustment.

16. The system of claim 15, wherein the scanning system obtains a single scan of a whole subject in motion.

17. The system of claim 15:
wherein the scanning system obtains a first slice scan of the subject in motion, the first slice scan being a scan of less than a whole subject;
wherein the location module determines if the first slice scan exceeds a threshold indicating that an entire defined target area is visible in the first slice scan and calculates the three dimensional position of the subject in motion based on the first slice scan if it is determined that the entire defined target area is visible in the first slice scan;
wherein the scanning system obtains an additional slice scan if it is determined that the first slice scan does not exceed the threshold;
wherein the location module combines the first slice scan and the additional slice scan to provide a combined scan and determines if the combined scan exceeds the threshold;
wherein the scanning system and the location module repeatedly obtain and combine until it is determined that the threshold has been exceeded; and
wherein the location module calculates the three dimensional position of the subject in motion based on the combined scan when it is determined that the threshold indicating that the entire defined target area is visible is exceeded.

18. The system of claim 15, further comprising at least one nozzle used to administer the substance to the subject in motion,
wherein the location module calculates a nozzle adjustment factor based on the calculated three dimensional position of the subject in motion adjusts a position of the at least one nozzle based on the calculated nozzle adjustment factor.

19. The system of claim 18, wherein the location module calculates the timing adjustment and the nozzle adjustment factor based on one or more of the following: a velocity of a conveyor belt on which the subject is traveling (v_{b}); a time of flight (TofF) before the substance is delivered to the subject; a speed at which the substance is deliver (vₛ); a distance the at least one defined target region is from a nozzle delivering the substance (dₜₙ); and a width of the conveyor belt (w_{c}).

20. The system of claim 18, wherein the at least one nozzle administers the substance to the at least one defined target region of the subject at a time and position altered by the nozzle adjustment factor and/or the timing adjustment.

21. The system of claim 18, wherein the at least one nozzle comprise one or more nozzle banks.

22. The system of claim 15, wherein the subject is a bird and the at least one defined target region is one or more of a mucosa in one or more eyes of the bird, an area around one or more eyes of the bird, nostrils of the bird, mouth of the bird, and/or any orifice on a head of the bird that leads to the gut and/or respiratory track.

23. The system of claim 15, wherein the subject is any human or animal subject that is prone to movement.

24. A computer program product for accurately administering a substance to a subject in motion, the computer program product comprising:
computer readable program code to obtain one or more scans of the subject, the subject having at least one defined target region thereon for delivery of the substance;
computer readable program code to calculate a three dimensional position of the subject in motion based on the obtained one or more scans of the subject, the three dimensional position including X, Y, and Z coordinates defining the three dimensional position;
computer readable program code to calculate a timing adjustment based on the calculated three dimensional position of the subject in motion; and
computer readable program code to adjust a timing of the delivery of the substance to the at least one defined target region on the subject using the calculated timing adjustment.

25. The computer program product of claim 24, wherein the computer readable program code to obtain comprises:
computer readable program code to obtain a first slice scan of the subject in motion, the first slice scan being a scan of less than a whole subject;
computer readable program code to determine if the first slice scan exceeds a threshold indicating that an entire defined target area is visible in the first slice scan;
if it is determined that the entire defined target area is visible in the first slice scan, computer readable program code to calculate the three dimensional position of the subject in motion based on the first slice scan;
if it is determined that the first slice scan does not exceed the threshold, computer readable program code to obtain an additional slice scan;
computer readable program code to combine the first slice scan and the additional slice scan to provide a combined scan;
computer readable program code to determine if the combined scan exceeds the threshold;
computer readable program code to repeat the obtaining and combining steps until it is determined that the threshold has been exceeded; and
computer readable program code to calculate the three dimensional position of the subject in motion based on the combined scan when it is determined that the threshold indicating that the entire defined target area is visible is exceeded.
